(19) United States
(12) Patent Application Publication
(10) Pub. No.: US 2022/0373566 A1
(43) Pub. Date: Nov. 24, 2022

(54) COAGULATION ASSAY APPARATUS AND METHODS THEREOF

(71) Applicant: Nova Biomedical Corporation, Waltham, MA (US)

(72) Inventors: Mary Pomeroy, Boynton Beach, FL (US); Martina Medkova, Winchester, MA (US); Jeff Chien, Wellesley, MA (US)

(21) Appl. No.: 17/767,143

(22) PCT Filed: Oct. 17, 2019
(86) PCT No.: PCT/US19/56676

§ 371 Date: Apr. 7, 2022

(51) Int. Cl. G01N 33/86; G01N 15/06

(52) U.S. Cl. CPC G01N 33/86; G01N 15/06; G01N 2015/0693; G01N 2015/0065

(57) ABSTRACT

This invention relates to a method and apparatus for determining the activity of coagulation factors in dilute capillary whole blood, citrated whole blood and citrated plasma. It also includes the detection of the hemoglobin amount in a whole blood sample so a correction of the clotting time can be performed thereby making the clotting time values independent of hemoglobin and hematocrit effect.

### BACKGROUND OF THE INVENTION

### 1. Field of the Invention

The present invention relates generally to a method and apparatus for determining the activity of coagulation factors in dilute capillary whole blood, citrated whole blood, and citrated plasma.

### 2. Description of the Prior Art

Methods and apparatus for determining the activity of coagulation factors in dilute capillary whole blood, citrated whole blood, and citrated plasma often involve a physician measuring a patient's International Normalized Ratio (INR) level during a Prothrombin Time-International Normalized Ratio (INR) test. This type of bioanalysis is designed to measure how much time it takes for a patient's blood to clot. This test ensures that patients are receiving the dosage and types of medications which prevent blood clots from forming and causing deep vein thrombosis (DVT), pulmonary embolism (PE), stroke, heart attack, and more. Such medications work by blocking the formation of vitamin K-dependent clotting factors, substances in the blood that cause clotting. If an INR score is too low, a patient can be at risk for a blood clot. However, if the INR is too high, patients could also experience bleeding. A typical INR score ranges between 2 to 3. The “ideal” INR score can vary from patient to patient.

How often a patient should be tested can vary, depending on how stable their INR is over time. According to the American Heart Association (AHA), patients should be tested at least once a month and, in some cases, as much as twice a week. This testing often involves going to get blood drawn and analyzed with in-vitro diagnostic analyzers.

In-vitro diagnostic analyzers have been available for several decades. The market for these types of analyzers were typically for use in a central laboratory. The central laboratory was capable of testing for a wide variety of biomedical species typically in a patient's blood and/or blood plasma. Lately, there appears to be an on-going shift for such testing from central laboratory testing to point-of-care sites within a hospital. This shift provides for quicker test data results, which can be important in diagnosis and treatment of certain conditions.

Point-of-care testing plays an important role in the management of critically ill patients and is widely used in the operating room, emergency room and intensive care units. These tests are no longer performed exclusively by skilled medical technologists but also by multiskilled personnel including nurses, respiratory therapists, emergency personnel, physicians, and other medical staff. To meet this demand, manufacturers have had to downsize the analyzers and simplify the test procedures so that only minimal training in performing the test procedures is required.

One key feature common to all point-of-care analyzers is that they must be either portable and/or transportable. Examples of such point-of-care analyzers include, but are not limited to, Opti CCA and Omni 9 critical care analyzers from Roche Diagnostics, a division of Hoffman-La Roche, Stat Profile Ultra C from Nova Biomedical Corporation, CRT from Nova Biomedical Corporation, and Dimension RxL from Dade Behring, Inc., a division of Siemens Healthcare Diagnostics.

More recently, there is a further shift occurring to testing in a physician's office or laboratory located within a physician's office. As testing moves away from the central laboratory, new single use medical devices have been developed to meet this need.

In the physician's office environment, there are numerous devices that utilize a capillary to collect finger stick samples for analysis. The capillary may be either glass or plastic. Typical analyses are for species such as HbA1c, lipids, etc. Once the sample is collected, these capillary-based collection devices are loaded into an analytical cartridge, which is then loaded into an instrument for analysis. Two known bioassays employed for diagnostic purposes will now be discussed with reference to their originating patent documents.

First, United States Patent Publication No. US 2011/0196085A1 discloses a stabilizing bead comprising a latex particle having a carboxylate group, and a stabilizing agent functionally coupled to the latex particle, wherein the stabilizing agent is capable of completely or substantially preventing the degradation or inactivation of a diagnostic agent in close proximity to the stabilizing agent. The stabilizing bead may further comprise at least one of human serum albumin (HSA), bovine serum albumin (BSA), or a linker group coupled to latex particle.

Second, European Patent No. EP0655627 discloses a method and test kit to perform a simple detection assay for D-dimer, a fibrin breakdown product, which utilizes purified Fragment E of human fibrinogen attached to a solid phase for direct chemical binding of D-dimer from a biological sample. Fragment E may be conjugated to latex carrier particles and an agglutination assay performed.

### SUMMARY OF THE INVENTION

Advantages and Differences of Invention Over Known Prior Art

The above-described parts of the prior art have not proven fully satisfactory for meeting all of the requirements of the industry. Inaccuracies have been found to be associated with the automated measurement of mean cell hemoglobin concentration in dehydrated cells. In coagulation, additional mixing and time for hydration etc., can induce uncontrolled procoagulant activation. Utilizing a dry, liquid, or dry and liquid reagent approach according to the present invention provides vast increased capabilities for particle and reagent assays over prior art methods.

The present invention employs carbohydrate matrixes to preserve the functionality of specific microparticles in order to increase capabilities for immunodiagnostics which prior to the present invention has been accomplished with varying amounts of success. Specifically, the present invention employs latex microparticles that have not been reacted, exposed, or coupled to proteins. These unreacted particles retain their bioactive adsorption properties when dried in carbohydrate matrices or used in a liquid reagent system. Upon use, the particles are capable of adsorbing biomolecules. This action facilitates and enhances the reactivity of the particle surface to the procoagulants contained in whole blood and plasma especially when used in a dilute sample/diluent environment. Included in this process is presentation of the reactive protein and particle mixture and to the various activation agents used in coagulation assays. Latex microparticles are employed in turbidimetric bioassays of the present invention in which the typical optical property of the sample solution is clear and not turbid.

The present invention addresses the failure of the prior art by providing a new application of a modified methodology to a dilute, lysed whole blood sample or plasma matrices where the latex particles provide the method of clot detection. Such a method allows the binding adsorption application to be applied at the point of clinical testing, reducing production time, cost and at the end user stage incubation reactivity time, and instrument footprint or size. Both untreated latex microparticles and particles with surface groups such as sulfate or amidine are effective for this invention. Appropriate buffer and thermal conditions are also necessary for each microparticle type to work in the dilute blood assay scheme.

Together, the biomolecule, microparticles, specialized buffer and assay temperature lend to rapid protein absorption for use in a rapid clinical assay. Retaining this property is paramount for functionality. Additionally, particle dispersion also has a major role. Uniform dispersion in solution permits rapid reaction and consistent analytical quantification of agglutination. Therefore, retaining both of these properties is mandatory for development of any reliable test component.

This invention provides a process methodology for drying uncoated, unconjugated, protein free, latex microparticles with plain or surface functionalized groups on polystyrene. Dispersed particles allow adsorption of quantifiable analytical protein biomarkers which are then available for ligand attachment. In particular, a matrix consisting of a carbohydrate facilitates adsorption of biomarker proteins while concomitantly allowing rapid dissolution and uniform dispersion of the particles.

It is an object of the present invention to provide a liquid microparticle reagent for ease in use for certain bioassays. It is another object of the present invention to provide a methodology for the employment of the dried or liquid latex microparticles in a diagnostic coagulation product. It is a further object of the present invention to provide a methodology for concurrent hemoglobin detection and quantitative correction of the clotting time value.

It is yet another object of the invention to provide a new method for the evaluation of the extrinsic coagulation pathway and the monitoring of oral anticoagulant therapy (OAT).

Methods of the present invention provide an assay for coagulation time of whole blood or plasma, with hemoglobin measurement and correction for whole blood samples. Various embodiments employ adjustments to the dilution level, temperature, type of particle and buffer components to alter the overall bioassay time. These embodiments employ a flexible dry, liquid, or, dry and liquid matrix along with specific microparticles. It is to be understood however that all assay components can be adjusted to give the most representative timing schemes for clotting bioassays according to the present inventive method. In general, the final whole blood or plasma sample dilution ratio should be in the range of 1 part in 50 to 75 for clotting assays performed between 33 C and 38 C.

The present invention achieves these and other objectives by providing a disposable bioassay diagnostic cartridge for monitoring anticoagulant activity. The disposable cartridge may have a first well holding an amount of matrix, the matrix being either a drying matrix or a liquid matrix; a second well holding microparticles. The microparticles may be uncoated latex with at least one surface type: unreacted plain, sulfate, carboxylate, and amidine chemical structures retaining activity. The cartridge may further have a third well with an amount of activation agent, the activation agent may be thromboplastin, thrombin, ellagic acid, activated partial thromboplastin, Factor II, Factor VII, Factor I, Factor X, Factor XII, activated protein C, snake venom, negatively charged phospholipids, calcium ions, tissue factor, silica, koalin, or celite.

The matrix may be a liquid carbohydrate matrix and or a drying matrix with at least one of NaCl, PEG, TWEEN, and CaCl₂). The disposable cartridge may have an integrated cuvette capable of facilitating dual optical detection readings. An integrated cuvette may have a first wall capable of facilitating a first optical detection reading via a first LED at 530 nm; and a second wall capable of facilitating a second optical detection reading via a second LED at 660 nm.

The present invention achieves other objectives by providing an all-inclusive coagulation bioassay diagnostic kit having all required components, excepting the analyzer itself. Such a kit may include a fingerstick, a pipette, bioassay components, and optical cuvette. To ensure sanitary delivery to the user, the fingerstick, pipette, bioassay components, and optical cuvette may be contained in a sanitary and sealed container having an identifier, such as a barcode which can be scanned by the analyzer. The bioassay components may have a matrix, and microparticles, where the microparticles may be uncoated latex with at least one surface type, and the at least one surface type being chosen from a group consisting of unreacted plain, sulfate, carboxylate, and amidine chemical structures retaining activity.

A further coagulation bioassay according to the present invention may have a carbohydrate matrix and microparticles within the carbohydrate matrix. This bioassay may also have an amount of activation agent, such as thromboplastin, thrombin, ellagic acid, activated partial thromboplastin, Factor II, Factor VII, Factor I, Factor X, Factor XII, activated protein C, snake venom, negatively charged phospholipids, calcium ions, tissue factor, silica, koalin, and celite. A matrix of this bioassay may either be a drying matrix or a liquid matrix, and a carbohydrate matrix may have maltrin 250, sucrose, or isomalt. The microparticles of a bioassay of this type may have a diameter of from about 10 nm to 150 nm and may be in a 1% weight per volume solution, 2% weight per volume solution, 4% weight per volume solution, 8% weight per volume solution, or 10% weight per volume solution.

By employing the bioassay above, the present invention seeks to provide a method of obtaining clotting time measurements for a blood sample type of any of dilute, lysed whole blood, whole blood (straight from a fingerstick), plasma, citrated blood, and/or mixed blood and plasma. Such a method would then include the steps of: selecting a microparticle matrix having a carbohydrate matrix and a plurality of microparticles within the carbohydrate matrix. The microparticles are preferably uncoated latex with at least one surface type when dried or liquid in the carbohydrate matrix, and the at least one surface type is preferably chosen from a group consisting of unreacted plain, sulfate, carboxylate, and amidine chemical structures retaining activity. This microparticle matrix may then be used as a reagent with the blood sample; and clotting time measurements of the one of dilute, lysed whole blood, or plasma may then be obtained through optical detection INR. Alternatively, a separate reagent may also be added to the reaction mixture to activate the natural clotting substrates in the blood sample. The separate reagent would preferably be an activation agent such as thromboplastin, thrombin, ellagic acid, activated partial thromboplastin, Factor II, Factor VII, Factor I, Factor X, Factor XII, activated protein C, snake venom, negatively charged phospholipids, calcium ions, tissue factor, silica, koalin, or celite.

Clotting time measurements may then be corrected for the hemoglobin concentration of the samples by simultaneously obtaining optical density readings at two different wavelengths.

### BRIEF DESCRIPTION OF THE DRAWINGS

FIG. 1 is an illustration of a method of the present invention.

FIG. 1a is an illustration of a user having selected a bioassay cartridge kit of the present invention for use with a scanner as shown.

FIG. 2 is an illustration of a user scanning a identifier of a cartridge of the present invention.

FIG. 3 is an illustration of a user using a fingerstick to obtain a blood sample according to a method of the present invention.

FIG. 4 is an illustration of a user retrieving a sampler from a cartridge of the present invention.

FIG. 5 is an illustration of a user filling a sampler from a cartridge of the present invention with a blood sample.

FIG. 6 is an illustration of a user replacing a filled sampler back to a cartridge of the present invention with a blood sample.

FIG. 7 is an illustration of a user placing the cartridge of the present invention with the filled sampler into the analyzer.

FIG. 8 is a cross-sectional view of a cartridge according to one embodiment of the present invention.

FIG. 9 is a cross-sectional view of a cartridge according to a further embodiment of the present invention.

FIG. 10 is a graphical representation of automated steps according to a method of one embodiment according to the present invention.

FIG. 11 is a graphical representation of a further look at one step of the method shown in FIG. 1.

FIG. 12 is a graphical representation of a further look at the step of the method shown in FIG. 11.

FIG. 13 is a graphical representation of a further look at another step of the method shown in FIG. 1.

FIG. 14 is a graph illustrating the effect of hemoglobin on Prothrombin Time against INR values with and without correction according to the present method.

FIG. 15 is a graph illustrating the effect of hemoglobin on Delta Prothrombin Time against INR values with and without correction according to the present method.

Prothrombin with BSA v. Normal

FIG. 16 is a graph illustrating a bioassay using microparticles with and without bovine serum albumin (BSA) surface groups on a normal sample.

FIG. 17 is a graph illustrating a bioassay using microparticles with and without bovine serum albumin (BSA) surface groups on an abnormal sample.

Prothrombin with Amidine v. Sulfate v. Plain

FIG. 18 is a graph illustrating the results of a prothrombin time assay for a first embodiment employing a first amidine particle dilution and a first matrix by measuring optical density at 660 nm against time.

FIG. 19 is a graph illustrating the results of a prothrombin time assay for a second embodiment employing a first sulfate particle dilution and a first matrix by measuring optical density at 660 nm against time.

FIG. 20 is a graph illustrating the results of a prothrombin time assay for a second embodiment employing a first surface free microparticle dilution and a first matrix by measuring optical density at 660 nm against time.

FIG. 21 is a graph illustrating the results of a prothrombin time assay for a fourth embodiment employing a second amidine particle dilution and a second matrix by measuring optical density at 660 nm against time.

FIG. 22 is a graph illustrating the results of a prothrombin time assay for a fifth embodiment employing a second sulfate particle dilution and a second matrix by measuring optical density at 660 nm against time.

FIG. 23 is a graph illustrating the results of a prothrombin time assay for a sixth embodiment employing a second surface free microparticle dilution and a second matrix by measuring optical density at 660 nm against time.

Prothrombin with Simethicone

FIG. 24 is a graph illustrating the results of a bioassay employing a sulfate microparticle dilution according to the present invention for normal whole blood by measuring optical density at 660 nm against time.

FIG. 25 is a graph illustrating the results of a bioassay employing a sulfate microparticle dilution according to the present invention for abnormal whole blood by measuring optical density at 660 nm against time.

FIG. 26 is a graph illustrating the determination of hemoglobin values present in the bioassay of FIG. 25, according to the present invention by measuring optical density at 520 nm.

FIG. 27 is a graph illustrating correction of the raw prothrombin time calculated in FIG. 25, as the result of adjustment by the determined hemoglobin value from FIG. 26.

FIG. 28 is a graph illustrating the determination of the INR value of the bioassay from FIG. 25 based upon the adjusted prothrombin time provided in FIG. 27.

Prothrombin with Carboxyl

FIG. 29 is a graph illustrating the results of a bioassay employing a carboxyl microparticle dilution according to the present invention for normal whole blood by measuring optical density at 660 nm against time.

FIG. 30 is a graph illustrating the results of a bioassay employing a carboxyl microparticle dilution according to the present invention for abnormal whole blood by measuring optical density at 660 nm against time.

Prothrombin with Varying Temperatures

FIG. 31 is a graph illustrating the results of prothrombin time assays for two embodiments of the present invention employing differing temperatures by measuring optical density at 660 nm against time.

FIG. 32 is a graph illustrating the results of prothrombin time assays for two embodiments of the present invention employing differing temperatures by measuring optical density at 660 nm against time.

Prothrombin with Coumadin

FIG. 33 is a graph illustrating and comparing results of bioassay of normal blood against bioassay of blood with Coumadin present by measuring optical density at 660 nm against time.

Thrombin

FIG. 34 is a graph illustrating and comparing the results of a thrombin time bioassay normal citrated plasma against thrombin time bioassay normal citrated blood by measuring optical density at 660 nm against time.

Activated Partial Thrombin

FIG. 35 is a graph illustrating the results of an activated partial thromboplastin time bioassay normal control.

FIG. 36 is a graph illustrating, for comparison, the results of the activated partial thromboplastin time bioassay with an abnormal control.

FIG. 37 is a graph illustrating the results of a bioassay for the activated partial thromboplastin time with 1stage factor assay for factor VIII with normal plasma by measuring optical density at 660 nm against time.

FIG. 38 is a graph illustrating and comparing the results of bioassays for the activated partial thromboplastin time with 1stage factor assay for factor VIII comparing APTT abnormal plasma and APTT 1-Stage mix by measuring optical density at 660 nm against time.

### DETAILED DESCRIPTION OF THE INVENTION

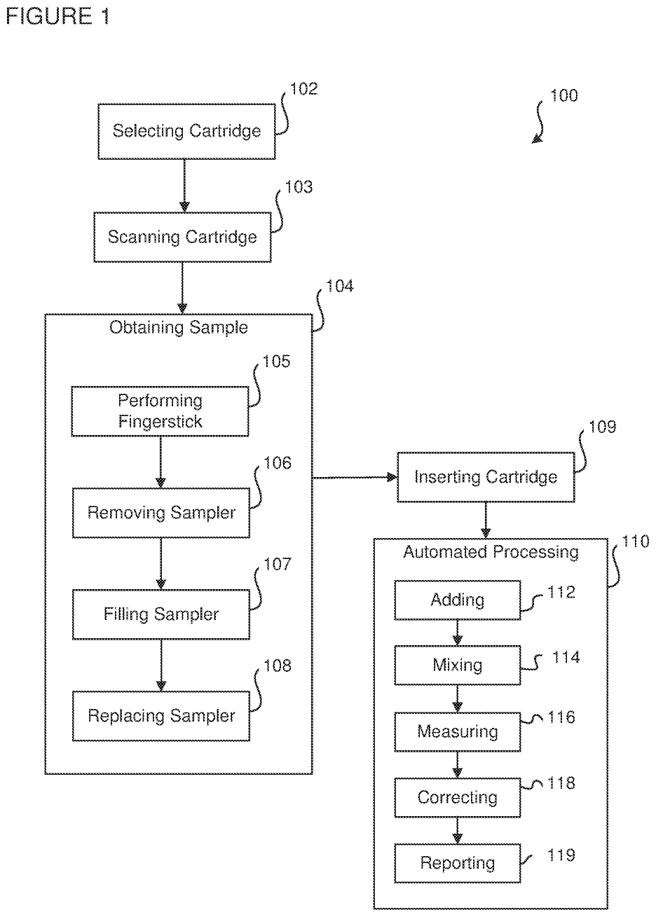

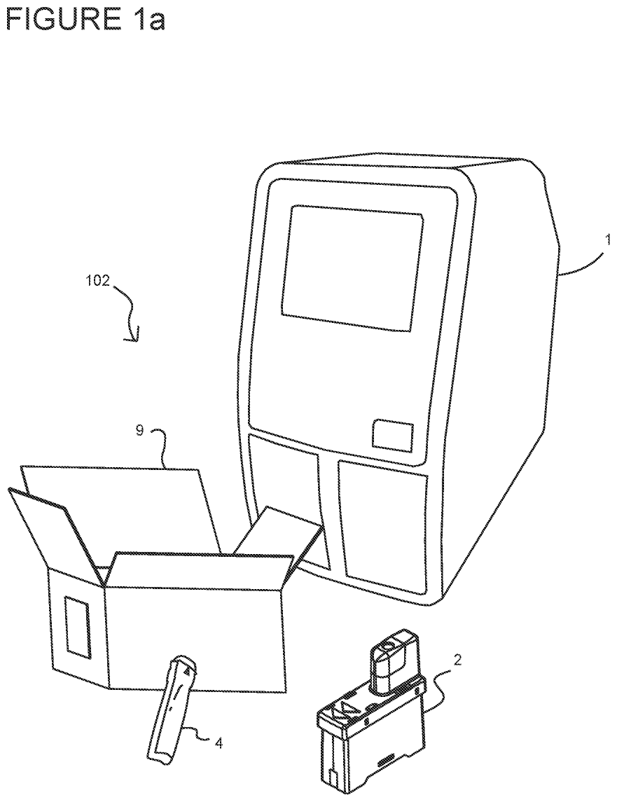

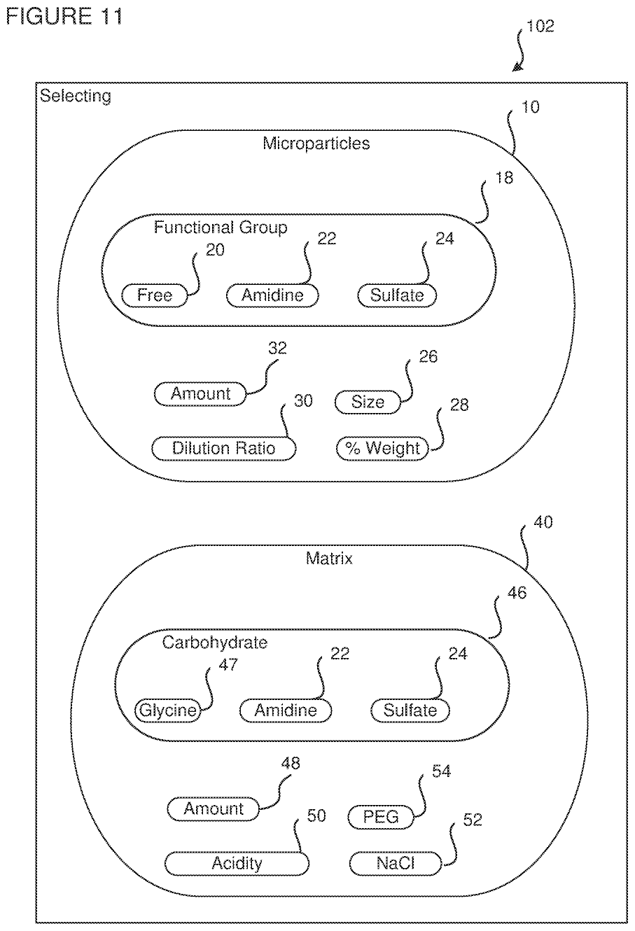

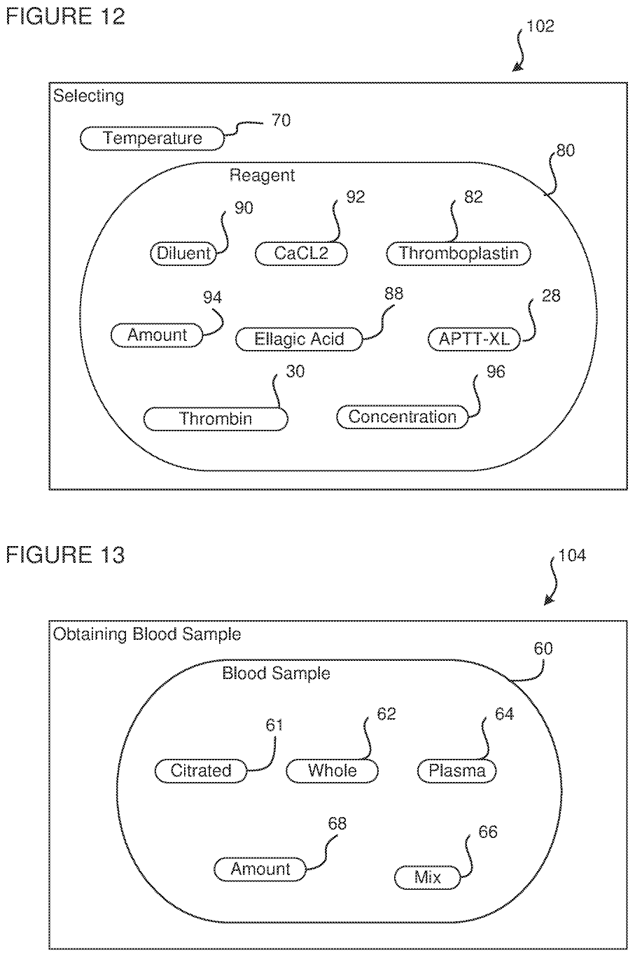

The preferred embodiments of the present invention are discussed in reference to FIGS. 1-38. As discussed above, the present invention provides a process, methodology, system, and apparatus relating to a method and apparatus for determining the activity of coagulation factors in dilute capillary whole blood, citrated whole blood, and citrated plasma.

### General Overviews

General overviews of the manual and automated portions of the overall method 100, system cartridge 2, and bioassay from various perspectives will now be discussed with indicated reference to FIGS. 1-12.

General Overview of Method from User's Perspective

A general overview of a user's perspective is now discussed with reference to FIGS. 1-7. As shown here, initially, a bioassay cartridge or cartridge kit is selected by the user 102. After manually selecting a bioassay cartridge 102, the cartridge may be scanned by the analyzer to identify the selected bioassay 103. Then, obtaining the blood sample 104 may involve a simple fingerstick 105 to provide the required blood sample size. The ease by which a blood sample may be obtained in this step exhibits one of the benefits of the present inventive system over other prior art systems and methods which require venipuncture or other large blood sample size collection.

If the bioassay is identified initially, the capillary sampler may be removed from the cartridge 106, filled with the blood sample 107, and replaced in the specified cartridge 108, all within five seconds of performing the fingerstick 105. After replacing the filled sampler 108 back in the cartridge, the identifier may be scanned 103 by the analyzer, and the cartridge inserted into the analyzer 109. After loading the cartridge into the analyzer, the automated process 110 begins. Depending upon the selected bioassay, the automated process will then, according to the specified order of the bioassay components, involve automated steps of adding 112, mixing 114, measuring 116, incubating 117, correcting 118, and reporting 119 the results to the user or other designated individual.

Depending on the system involved, and the purpose for the bioassay, the automated process 110 may include a further step of automatically adjusting 120 the amount of medication prescribed and/or the amount of medication provided by a built-in medication delivery system (not shown).

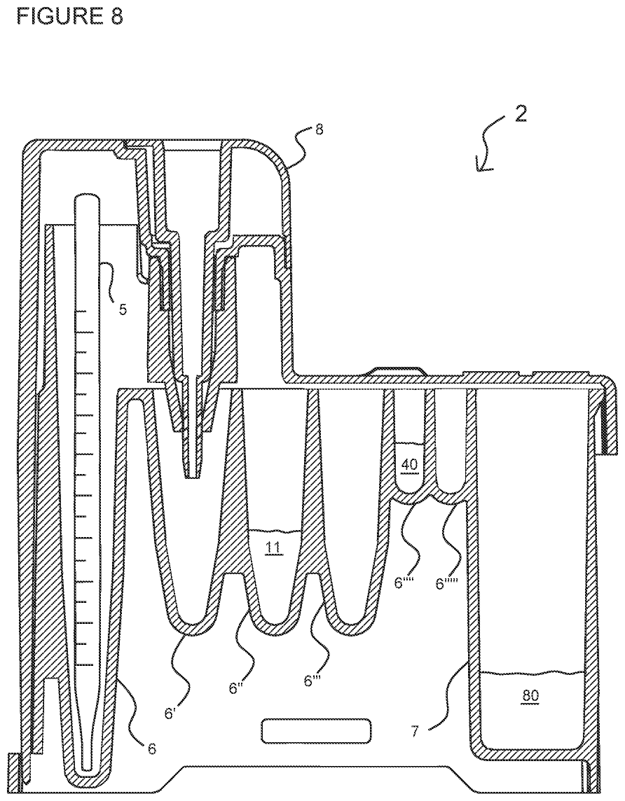

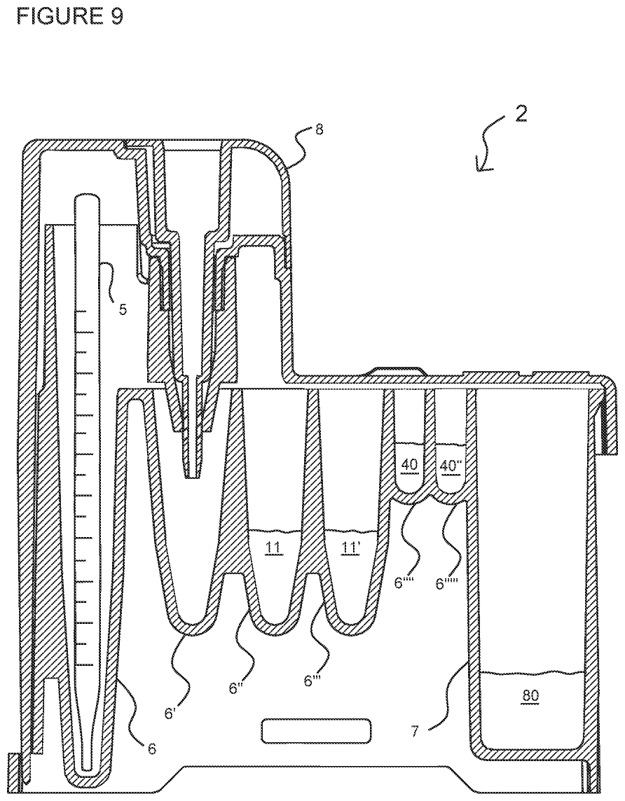

Shown in FIG. 1A is an illustration of a cartridge kit after having been selected 102 by a user. This cartridge kit includes a bioassay cartridge 2 and fingerstick 4 prepackaged for singular use with an analyzer system 1. The cartridge kit preferably also includes sterilized pipettes 5, samplers 8, and a cuvette 7 which are discussed in greater detail below with respect to FIGS. 8-9. An exemplary analyzer system 1 capable of performing the bioassay as described herein is the ALLEGRO™ analyzer by NOVA Biomedical Corporation. An exemplary sampler and cartridge base capable of being used with a cartridge 2 as described herein is more fully described in U.S. Pat. No. 10,117,615 of NOVA Biomedical Corporation.

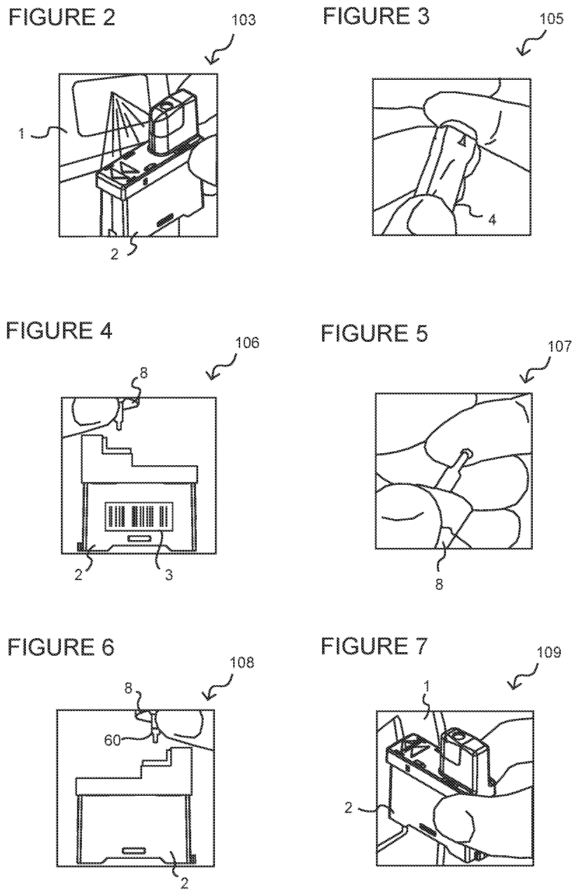

Turning next to FIGS. 2-7 which further illustrate the use of the specific components of the cartridge kit from a user's perspective. Specifically, FIG. 2 illustrates a user having the analyzer 1 identify the bioassay method by scanning the selected cartridge 2 of the present invention. Then, FIG. 3 illustrates the user obtaining access to a blood sample 60 by using the fingerstick 4 provided with the cartridge kit. FIG. 4 then shows the user retrieving a sampler 8 from the cartridge 2. Next, FIG. 5 shows the user filling that sampler 8 with the blood sample 60 accessed by the fingerstick 4. Afterwards, FIG. 6 illustrates the user replacing the filled sampler 8 back in the cartridge 2 of the present invention with the blood sample 60 obtained from the fingerstick 4. Finally, FIG. 7 shows the user placing the cartridge 2 with the filled sampler 8 into the analyzer 1, thereby initializing the selected and identified bioassay. By providing these components as a unified kit in a single package 9, the present invention both reduces overall procedure time and reduces user error.

General Overview of the Bioassay Cartridge

An exemplary cartridge kit which may be used with the present inventive system and methodology may include a self-contained single-use disposable integrated bioassay cartridge 2 such as now will be described with further reference to FIGS. 8, 9.

A first cartridge 2 embodiment, illustrated in FIG. 8, is prepared with bioassay components for only a single type of bioassay according to one embodiment of the present invention. Alternatively, the second cartridge embodiment, illustrated in FIG. 9, is a multi-purpose cartridge 2′ which is pre-loaded with multiple components for at least two types of bioassays according to further embodiments of the present invention.

Both FIGS. 8 and 9 illustrate cartridges 2 having an identifier 3, such as a barcode, capable of identifying the specific type of cartridge (and thus bioassay) to the analyzer 1. This identifier 3 may be visible along a visible outer surface of the cartridge 2, or along an outer surface of a main packaging 9. This packaging 9 may contain a cartridge 2, fingerstick 4, sterilized pipettes 5, sampler 8, and cuvette 7. Both FIGS. 8 and 9 illustrate cartridges 2 in which the capillary sampler 8 is a removable component of the cartridge 2 itself. Both FIGS. 8 and 9 also illustrate a cartridge 2 having an integrated cuvette 7, with sidewalls capable of facilitating optical measurement. However, it is also anticipated that the packaging 9 may contain a cartridge which requires a separate disparate cuvette 7 provided in addition to the cartridge.

Both FIGS. 8, 9 also illustrate cartridges 2 having a series of wells 6 which are pre-loaded with components of the assay according to the present inventive method. With both types of cartridge, the selecting stage 102 (discussed above with reference to FIGS. 1-7) involves selecting the desired bioassay cartridge. After selecting the desired cartridge/assay 102, the cartridge is scanned 103, filled 107, and then inserted into the system 109.

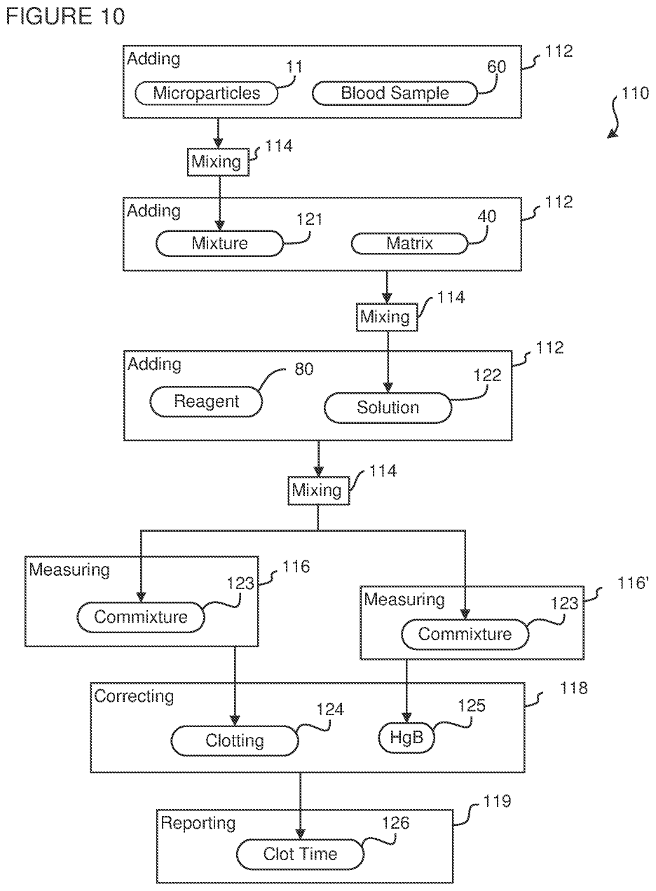

From a user's perspective, the only difference between using the first and second type of cartridges begins now. With the first cartridge, a user may then manually select an automated process option 110 by selecting one of several bioassay options through manual user input via an access control panel on the analyzer. Whereas, with the second cartridge type, insertion of the cartridge 109 alone is sufficient for triggering the automated process 110. For this second cartridge type, from a user's perspective, the bioassay cartridge alone is ‘selected’. However, before a user can select a bioassay option, the bioassay cartridge must first be prepared, and before preparation, the bioassay components themselves must first be selected. Several bioassay cartridge component options will be discussed further below with reference to the initial FIG. 1, and further with respect to FIGS. 10-13.

Now looking more specifically at those elements of the cartridge 2 which are not necessarily observed by the user once the sampler 8 has been filled 107 and returned to the cartridge 2. As may be seen in FIGS. 8, 9 (although not numbered), the sampler 8 has a capillary element which is inserted through a corresponding capillary-receiving aperture in an extension portion top surface of a stepped extension portion of a cover extension of the disposable test cartridge 2 and is then seated in the stepped extension portion.

During the insertion and setting process, the capillary tube of the sampler 8 is inserted through the lower portion aperture located in an apex end of the capillary wiper. Because the cross-sectional area of the lower portion aperture is smaller than the cross-sectional area of the capillary tube, the lower portion aperture acts like a squeegee against the outside surface of the capillary tube and prevents any sample inadvertently disposed on the outside surface of the capillary tube from entering and being deposited into the chamber 6 of the cartridge 2.

Also facilitating accuracy, the capillary wiper of these cartridges 2 removes any sample 60 from the outside surface of the capillary tube. Erroneous results are thereby prevented from an “over-filling” of the appropriate well 6′ in the test cartridge 2 with sample 60. Likewise, since the capillary tube is not wiped by the user, there is no, or very little, chance that any sample 60 within the capillary tube is removed inadvertently, which could lead to erroneous results from an “under-filling” of the well 6′ in the test 2 cartridge with sample 60.

General Overview of Automated Processes

The cartridge 2 is then inserted into the point-of-care analyzer 1 for the automated testing portions 110 of the blood sample 60 which will now be discussed with further reference to FIG. 10. Once inside the analyzer 1, an automated arm of the analyzer disconnects a cover of the cartridge 2 by unlocking releasable tabs, the cover having the capillary sampler 8 and sample 60 still enclosed within.

The analyzer then uses a small a small sharp point on the cartridge cover to pierce seals for each of the wells 6, 6′, 6″, etc. Seals of the present cartridge 2 may be foil seals, or other covers, so long as they are capable of preventing cross contamination of the contents of the wells during shipping. These seals should also be capable of reducing unregulated water vapor dilution and evaporation.

The automated arm then retrieves a pipette tip 5 from the first well 6. The pipette tip 5 is then used by the analyzer 1 to move the samples and other bioassay components to the appropriate well for mixing according to the bioassay methods described herein.

If a dry microparticle matrix 11 is employed, then the pipette 5 aspirates additional diluent 90 from a fourth well 6′″ and adds it according to the protocol to microparticles 11 within the third well 6″, where mixing and stirring occurs forming diluted particles 11. Then, regardless of which type of matrix is employed, the pipette tip 5 aspirates the selected amount of (now diluted) microparticles 11 from the third well 6″ into a second well 6′.

The cover of the cartridge 2 is then replaced on the cartridge and the pipettor engages the capillary sampler 8 and the sample 60 is thereby added 112 to the second well 6′ where the diluted selected microparticles 11 await. The arm of the analyzer 1 then removes the cover of the cartridge 2 again and the sample 60 and diluted selected particles 11 are then mixed 114 by pipetting up and down, for a first predetermined time forming a mixture 121 of microparticles 11 and sample 60.

The pipette tip 5 then aspirates the designated amount of the selected, formed matrix 40 from the fifth well 6″″ then, in a second adding step 112, adds the matrix 40 to the second well 6′ which already comprises the mixture 121 of sample 60 and diluted microparticles 11. The matrix 40, and the mixture 121 are then mixed 114, for example, by pipetting up and down, within the second well 6′ for a second predetermined time forming a solution 122 (having matrix 40, sample 60, and microparticles 11).

Afterwards, the pipette 5 aspirates a predetermined amount of the solution 122 (comprising the matrix 40, sample 60, and microparticles 11) from the second well 6′ and adds 112 this solution 122 to a seventh well 7 in the cartridge 2 comprising the selected reagent 80. The solution 122 (microparticles, the matrix, the sample) and the reagent 80 are then mixed for a third predetermined time forming a commixture 123 (now having matrix 40, sample 60, microparticles 11, and reagent 80).

If performed with a preferred cartridge 2 as shown in FIGS. 8 & 9, this seventh well 7 is also an integrated optical detection cuvette 7 having walls of sufficient clarity, viscosity, and thickness to ensure that optical detection can now occur without further displacement of the commixture 123. As will be discussed in more detail further below, the optical density of the commixture 123 will then be measured 112, 112′ at least twice.

Initiating the first measurement 112, a first light emitting device (LED) of the analyzer 1 is turned on, the light from the first LED is transmitted a wavelength of 660 nm through a first wall of the integrated cuvette 7. The light is then transmitted through the commixture 123, and through a second wall of the integrated cuvette until the light is then detected by a light detector of the analyzer. Continuous readings are collected over a predetermined amount of time to provide first clotting data 124.

For the second measurement 112′, a second light emitting device (LED) of the analyzer 1 is turned on, the light from the second LED is transmitted at a second wavelength of 530 nm through the first wall of the integrated cuvette 7. The light is then transmitted through the commixture 123, and back through the second wall of the integrated cuvette 7 until the light is then collected by a light detector of the analyzer. Only a single reading is necessary to provide the Hemoglobin level data 125. It is to be recognized that although these measurements are called first and second, the order may be reversed. Alternatively, instead of providing the measurements sequentially, they may be provided contemporaneously, or even partially concurrently.

As will be discussed further below, the results from the optical detection 116, 116′ are then used to correct 118 the clotting time results 126. It is these corrected results 126 which are then reported to the user.

General Overview of Bioassay Components

The present invention provides a bioassay cartridge 2 having at least one of several microparticle matrixes 10 which will now be discussed. In some embodiments, the microparticle matrix 10 is formed prior to cartridge formation, in other embodiments, the microparticle matrix 10 is formed after addition 112 of the blood sample 60 during the bioassay process 110.

The microparticle matrix 10 has microparticles 11 which may be uncoated, unconjugated, protein free, latex, plain, or with surface functionalized groups on polystyrene microparticles. The matrix 40 generally consists of a carbohydrate 46 which facilitates adsorption of biomarker proteins while concomitantly allowing rapid dissolution and uniform dispersion of the microparticles 11. The dispersed nature of the microparticles 11 within the matrix 40 allows adsorption of quantifiable analytical protein biomarkers which are then available for ligand attachment.

Microparticles 11 used in the present invention include polystyrene microparticles 18 with surface active groups such as amidine 22 and sulfate 24. The microparticles 11 of the present invention range in diameter size 26 from 20 nm to 800 nm or more. The preferred range of particle diameter 26 is 40 nm to 150 nm, with the most preferred diameter 26 being in a range of 75 to 125 nm.

Amidine Microparticles

One embodiment of the present invention employs amidine particles 22 having a diameter 26 of 95 nm in a dilution 11 of 0.080% weight per volume 28. The optical density 31 of the dilution 11 was measured at 660 nm, for an optical density value of 0.19.

The amidine microparticles 22 of the present invention range in diameter size 26 from 20 nm to 800 nm or more. The preferred range of particle diameter 26 is 40 nm to 150 nm, a more preferred diameter 26 being in a range of 75 to 125 nm; and the most preferred diameter being in a range of 90 nm to 98 nm. The amidine microparticles 22 are in a dilution 11 in a range of 0.006%-8% weight by volume 28, and more preferably in a dilution 11 having a range of 0.010%-0.20% weight by volume, and most preferably a dilution 11 having a 0.080% weight by volume.

The present bioassay methods using amidine latex particles 22 call for a dilution 11 having a total dilution ratio 30 of microparticles 22 to dihydrogen monoxide in ranges of 1:10 to 1:400; and more preferably in a range of 1:20 to 1:150.

Amidine latex particles 22 which may meet the objectives of the present inventive bioassay methods may include high activity latex beads provided from Invitrogen™ when prepared according to the present inventive methods discussed herein.

Sulfate Microparticles

Another embodiment of the present invention employs sulfate microparticles 24 having a diameter of 110 nm in an 0.044% weight per volume dilution. The optical density of the dilution 11 was measured at 660 nm, for a value of 0.21.

Other sulfate microparticles 24 of the present invention range in diameter size 26 from 20 nm to 800 nm or more. The preferred range of sulfate particle 24 diameter 26 is 40 nm to 150 nm, a more preferred diameter 26 being in a range of 75 to 125 nm; and the most preferred diameter 26 being in a range of 90 nm to 110 nm.

According to some embodiments, sulfate microparticles 24 are in a dilution 11 in a range of 0.001% to 12% weight by volume, and more preferably in a dilution 11 having a range of 0.01% to 8% weight by volume, and most preferably a dilution 11 having a 0.016% weight by volume.

The present bioassay methods using sulfate microparticles 24 call for a dilution ratio 30 having a ratio of microparticles 24 to dihydrogen monoxide in ranges of 1:50 to 1:2000; more preferably a ratio in a range of 1:100 to 1:1000; and most preferably in a ratio of 1:500.

Sulfate latex particles 24 which may meet the objectives of the present invention may include high activity latex beads provided from Invitrogen™ when prepared according to the present inventive methods discussed herein.

Surface Free Microparticles

Another embodiment of the present invention employs surface free microparticles 20 having a diameter 26 of 96 nm in a dilution 11 having a 0.067% weight per volume 28. This dilution has a dilution ratio 30 of 1 to 150. The optical density 31 of the dilution 11 was measured at 660 nm, for a value of 0.21.

Other surface free microparticles 20 of the present invention range in diameter size 26 from 20 nm to 800 nm or more. The surface free microparticles 20 have a preferred range of diameter 26 of 40 nm to 150 nm, a more preferred diameter 26 being in a range of 75 to 125 nm; and the most preferred diameter 26 being in a range of 90 nm to 110 nm. According to some embodiments, surface free microparticles 20 are in a dilution 11 in a range of 0.001% to 2% weight by volume, and more preferably in a dilution 11 having a range of 0.01% to 0.2% weight by volume, and most preferably a dilution 11 having a 0.016% weight per volume.

The present bioassay methods using surface free microparticles 20 call for a dilution 11 having a dilution ratio 30 of microparticles 20 to dihydrogen monoxide 33 in ranges of 1:50 to 1:2000; and more preferably in a range of 1:100 to 1:1000.

Surface free particles which may meet the microparticles requirements of the present invention may include plain microparticles as provided from Varian Labs™, when prepared according to the present inventive methods discussed herein.

Carboxyl Microparticles

Other embodiments of the present invention employ carboxyl latex microparticles having a diameter of 103 nm in a 0.016% weight per volume solution. The optical density of the dilution 11 was measured at 660 nm, for a value of 0.08.

Other carboxyl microparticles 25 of the present invention range in diameter size 26 from 20 nm to 800 nm or more. The preferred range of carboxyl particle 25 diameter 26 is 40 nm to 150 nm, a more preferred diameter 26 being in a range of 75 nm to 125 nm; and the most preferred diameter being in a range of 90 nm to 110 nm.

According to some embodiments, carboxyl microparticles 24 are in a solution 28 in a range of 0.001% to 2% weight by volume, and more preferably in a solution 28 having a range of 0.005% to 0.1% weight by volume, and most preferably a dilution 11 having a 0.016% weight by volume.

The present bioassay methods using carboxyl microparticles 25 call for a dilution 11 having a dilution ratio 30 of microparticles 25 to diluent 33 in ranges of 1:50 to 1:2000; and more preferably in a range of 1:100 to 1:1000.

Carboxyl latex particles 24 which may meet the objectives of the present invention may include high activity latex beads provided from Invitrogen™ when prepared according to the present inventive methods discussed herein.

Drying Matrices

In general, the bioassay methods of the present invention can utilize most latex particle suspensions, plain or with functionalized surfaces. Some embodiments employ a matrix having reagent attenuators such as surfactants. Two such reagent attenuators which were tested include polysorbate-type nonionic surfactants and octylphenol ethoxylate surfactants. Polysorbate-type nonionic and octylphenol ethoxylate surfactants which may meet the objectives of the present invention may include surfactants provided by the Tween™ and Triton™ families when prepared according to the present inventive methods discussed herein.

Some of the drying matrices according to the present invention include carbohydrates, carbohydrate derivatives, and mixtures which create an environment which protect the particles from adverse temperatures and permit rapid re-hydration and uniform dispersal upon addition of fluid such as buffer, diluted sample, or other fluid reagent.

Carbohydrates, and their derivatives, are the preferred compounds used to dry the particles and provide stabilization during the drying process for the bioassay methods discussed herein. These reagents are prepared in water. However, low molarity buffers are also used in other embodiments, and examples of these include glycine and bicine. The percent concentration of the stabilizers range from 2 to 25% with a preferred range of 5 to 10%.

The compounds used for dry matrices include: glycine, bicine, sodium chloride, n-octenyl succinic anhydride, polyvinyl alcohol-polyethylene glycol graft copolymer, maltodextrins, α-(1,6)-linked maltotriose, α-D-glucopyranosyl-(1→1)-α-D-glucopyranoside, water-soluble glucose polymers obtained from the hydrolysis of starch with acid and/or enzymes in the presence of water, polysaccharide polymer, polyethylene glycols, polyethylene glycol (15)-hydroxystearate, povidones, sucrose, sorbitol, poly-oxyethylene esters of 12-hydroxystearic acid, and 1-O-alpha-D-glucopyranosyl-D-mannitol.

Publicly available versions of these compounds products which may meet the objectives of the present invention may include HiCap 100™, Kollicoat IR™, Maltrin 250™ Pullulan, Trehalose, Solutol™ Plus, and Solutol™. The preferred embodiment solutions are Lab 9101™, Maltrin 250, Trehalose™ and sucrose with the most preferred matrices being Maltrin™, sucrose, and isomalt. While for embodiments using dried sulfate microparticles, the preferred carbohydrate is sucrose or isomalt.

Liquid Matrices

The same particle suspensions described above for each of the dry matrixes may also be made in a more dilute form for ease in dispensing and aspiration using so called ‘liquid’ matrices. For example, when the dry matrix formulation consists of 25 uL of 1:18 sulfate latex with 10% sucrose, a similar liquid matrix has a reagent in the range of 50 uL of 1:9 sulfate latex with 5% sucrose. In other embodiments, the liquid matrix is formed of a lyse diluent.

Compounds employed in the liquid matrices of the present embodiments include: glycine, bicine, sodium chloride, n-octenyl succinic anhydride, polyvinyl alcohol-polyethylene glycol graft copolymer, maltodextrins, α-(1,6)-linked maltotriose, α-D-glucopyranosyl-(1→1)-α-D-glucopyranoside, water-soluble glucose polymers obtained from the hydrolysis of starch with acid and/or enzymes in the presence of water, polysaccharide polymer, polyethylene glycols, polyethylene glycol (15)-hydroxystearate, povidones, sucrose, sorbitol, poly-oxyethylene esters of 12-hydroxystearic acid, 1-O-alpha-D-glucopyranosyl-D-mannitol, polyethylene glycol (PEG), PEG 6K, PEG 12K, and PEG 20K, polysorbate-type nonionic surfactants, octylphenol ethoxylate surfactants, and/or simethicone diluted with water for injection grade purified water to a concentration supporting less than 15 second de-foaming performance in a USP Assay.

Publicly available versions of a select few of these compounds products which may meet the objectives of the present invention may include HiCap 100™, Kollicoat IR™, Lab 9101 ™; Maltrin 250™, Pullulan™, Trehalose™, Solutol Plus™, Solutol™, FoamAWAY™, and Sorbital™

Reagents

The bioassay methods of the present invention employ a variety of reagents 80 or activators. Compounds employed as activators of the present embodiments include: thrombokinase, thromboplastin, tissue thromboplastin factor III, platelet tissue factor, thrombokinase, thrombozyme, tissue factor, zymoplastic substance, ellagic acid, activated partial thromboplastin, thrombin, factor II, factor VII, factor I, factor X, factor XII, activated protein C, negatively charged phospholipids, calcium ions, alumina silicate clay, silicon oxide, silica, celite, and povidones.

Commercially available versions of a select few of these compounds and products which may meet the objectives of the present invention, when prepared appropriately, may include Kaolin™, Innovin™ Thromboplastin 82, APTT-XL 84, and Siemens™, Thrombin 86.

While stored for shipment prior to the actual bioassay, these reagents or activators may be in a dry state to ensure additional stability. Then during the assay, these reagents 80 may be diluted with diluent 90 such as DH₂O, etc, at various amounts 94 in a range of 20 uL to 400 uL, more preferably in a range of 50 uL to 300 uL, and in a range of 50 uL to 100 uL, in various concentrations 96. Still other embodiments employ CaCl₂) or other diluents which will be discussed with regard to the specific embodiments below.

Optical Density & Corrections

As initially discussed with regard to FIG. 10, the principle of coagulometric (turbidimetric) clot detection is used in the present invention and system to measure 116, 116′ and record the amount of time required for a plasma or whole blood sample 60 to clot. This technique assesses coagulation initiation 132 and coagulation endpoint 134 by measuring a change 135 in the optical density 130 detected over time.

Clot formation 124 is inferred and ‘detected’ based on the principle that in a medium in which fibrinogen is converted to fibrin, any light passing through this medium will be absorbed by the fibrin strands. Thus, as fibrin clot formation progresses over the passage of time, light absorption increases, resulting in a change 135 in the optical density 130.

For each of the bioassays discussed herein then, as discussed above, after collecting the sample, and mixing, light is transmitted from a source through the commixture 123. The transmitted light is then directed onto a light detector, which is positioned 180° incident to the source. The first corresponding electrical signal output from the photodetector of the first measurement 116′ uses optical detection to determine the levels of hemoglobin 125.

Light transmittance through the commixture 123 is again measured 116 by the photo detector for a predetermined time, which generates a second corresponding electrical signal output. Both this first and the second corresponding electrical signal outputs from the photo detector changes according to the detected light.

The signal output is processed via software through a series of algorithms to determine an associated clot point, clot initiation 132, and formation 134. In short, the change 135 in optical density 130 of the latex microparticle commixture 123 signal is used to indicate clot initiation 132 and formation 134.

Decreased Operational Procedure Elapsed Time

The present invention seeks to address several sources of laboratory error for coagulation detection procedures. As clot formation is dependent upon the passage of time, one of the largest sources of laboratory error for coagulation detection procedures is due to the passage of time between sampling 104 and measuring 116.

One of the principles of the present invention is to address the prior art's failure to address the increased number of errors which can occur due to the passage of time between sample taking 104 and detecting (measuring) 116. By pre-packaging the assay components in an all-inclusive cartridge 2 and providing pre-selected components for assay methodology, the present invention expedites the pre-detecting stages.

Another way in which the present invention achieves this goal is by providing a bioassay which is capable of achieving accurate results by using whole blood samples without the need to separate the red blood cells from the plasma before testing. Currently available point-of-care analyzers require the use of blood plasma as the sample. This requires separation of the red blood cells from the plasma in a blood sample before obtaining test results and further prolongs the period of time between the sample taking and testing.

One of the reasons current analyzers require blood plasma was discovered to be due to the inability of prior art automated systems and assays to provide self-correcting analysis. Levels of hemoglobin in the blood samples will have an impact upon the rate of clot initiation. The present inventors have found that failure to adjust the signal output of optical density to account for the variations in personal hemoglobin amounts can create errors in correctly identifying clot initiation and formation.

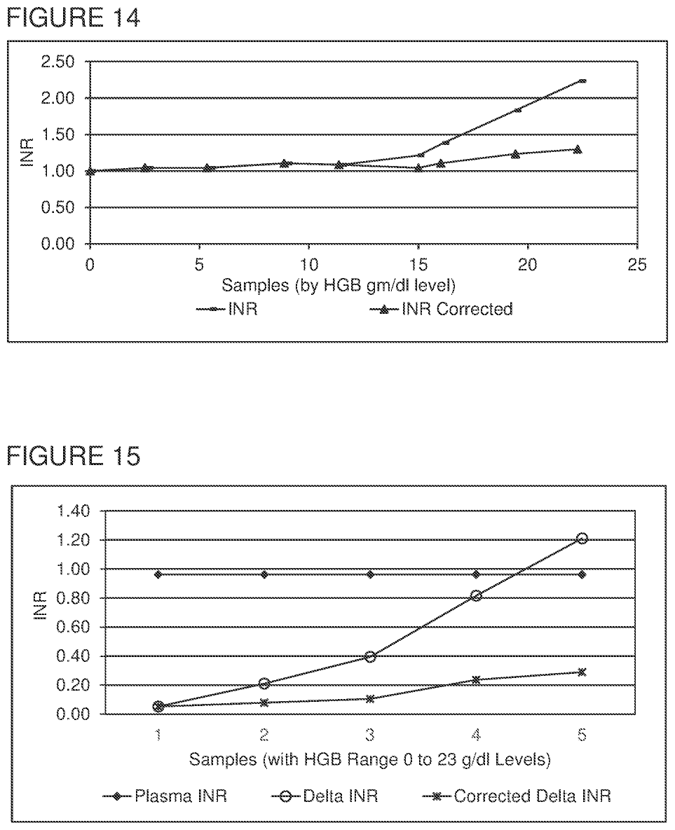

In order to address the errors of the prior art, with regard to this potential source of error, the present invention provides a bioassay method for auto-correction for hemoglobin. For the described clotting assay embodiment using whole blood, the optical density of the commixture is measured at a visible wavelength. Sequentially, the hemoglobin level is determined by measuring the sample optical density at another visible wavelength. The clotting time is then corrected using the hemoglobin measurement for the adjusted true plasma value of the sample. To illustrate the benefit of the advantages provided by this automatic correction, the effect of hemoglobin on prothrombin time INR values, with and without correction, is graphically illustrated in FIG. 14.

Specifically, the graph in FIG. 14 represents the effect of various levels of hemoglobin (hematocrit effect) on the INR of a normal sample. As shown here, the hemoglobin ranged from 0 to 23 gm/dl. Normal INR's range from 0.8 to 1.3 INR units. Moderate oral anticoagulation therapy results in INR's from 1.8 to 2.8 INR units. As the hemoglobin is increased, the INR is increased due to decreased plasma portion in the whole blood sample. With the additional LED in the visible range, the INR can be corrected for the hemoglobin detected. This figure also shows the corrected INR for hemoglobin in comparison.

The present invention facilitates the expansion of instrument capabilities to include multiple signal measuring devices such as, but not limited to, light emitting diodes (LED's) at multiple wavelengths. Measuring the optical density of the commixture to read the assay is preferably performed at a visible wavelength, in a range of 620 nm to 700 nm, more preferably between 650 to 680 nm, even more preferably between 658 nm and 668 nm, and most preferably at 660 nm. Measuring the optical density of the sample to read the hemoglobin levels is preferably performed at a visible wavelength, in a range of 500 nm and 550 nm, more preferably between 510 nm and 545 nm, even more preferably between 520 nm and 540 nm, and most preferably at 530 nm.

Table 1 shows the data which is graphically illustrated in FIG. 14, identifying the sample ID, hemoglobin levels, and initial INR before correction:

TABLE 1
HGB Effect on INR

| Sample | HGB mg/dl | INR |
|---|---|---|
| 1 | 0 | 1.00 |
| 2 | 2.6 | 1.04 |
| 3 | 5.5 | 1.04 |
| 4 | 9.0 | 1.11 |
| 5 | 11.5 | 1.09 |
| 6 | 15.0 | 1.21 |
| 7 | 16.1 | 1.38 |
| 8 | 19.4 | 1.83 |
| 9 | 22.4 | 2.23 |

Table 2 shows the data which is also graphically illustrated in FIG. 14, identifying the sample ID, hemoglobin levels, and initial INR after correction:

TABLE 2
HGB effect on INR Corrected

| Sample | HGB mg/dl | INR |
|---|---|---|
| 1 | 0 | 1.00 |
| 2 | 2.5 | 1.04 |
| 3 | 5.3 | 1.04 |
| 4 | 8.9 | 1.11 |
| 5 | 11.4 | 1.09 |
| 6 | 15.0 | 1.04 |
| 7 | 16.0 | 1.11 |
| 8 | 19.4 | 1.23 |
| 9 | 22.3 | 1.30 |

Next, shown in FIG. 15, the differences between the plasma INR value(s) obtained on a preapproved PT/INR analyzer are graphically illustrated with and without correction for hemoglobin according to the present method. Again, the hemoglobin values ranged from 0 to 23 g/dl.

Table 3 shows the data which is graphically illustrated in FIG. 15, identifying the sample ID, plasma INR, the difference (DELTA INR) from the plasma INR value before and after correction for hemoglobin.

TABLE 3
Effect of HGB on Plasma INR, Delta, Corrected

| Sample | Plasma INR | Delta INR | Corrected Delta INR |
|---|---|---|---|
| 1 | 0.96 | 0.05 | 0.05 |
| 2 | 0.96 | 0.21 | 0.08 |
| 3 | 0.96 | 0.39 | 0.11 |
| 4 | 0.96 | 0.82 | 0.24 |
| 5 | 0.96 | 1.21 | 0.29 |

Normalized Prothrombin Time/INR

It is to be understood that normalized INRs are employed in various embodiments of the bioassays of the present invention. Normalized prothrombin time INR are employed to address differences in the thromboplastins used which create variations in output. This INR correction measurement (or normalization) is developed from the prothrombin time, the sensitivity index of the thromboplastin, and the mean prothrombin time.

Specifically, in mathematical formulation, this normalization can be written as the following calculation:

INR=(PT/MT)^{ISI}

Where in the above formulation, INR stands for the normalization value; PT stands for the Prothrombin test time; ISI stands for the sensitivity index of the thromboplastin; and MT stands for the mean prothrombin time derived from 20 normal samples.

Specific Bioassays of the Present Invention

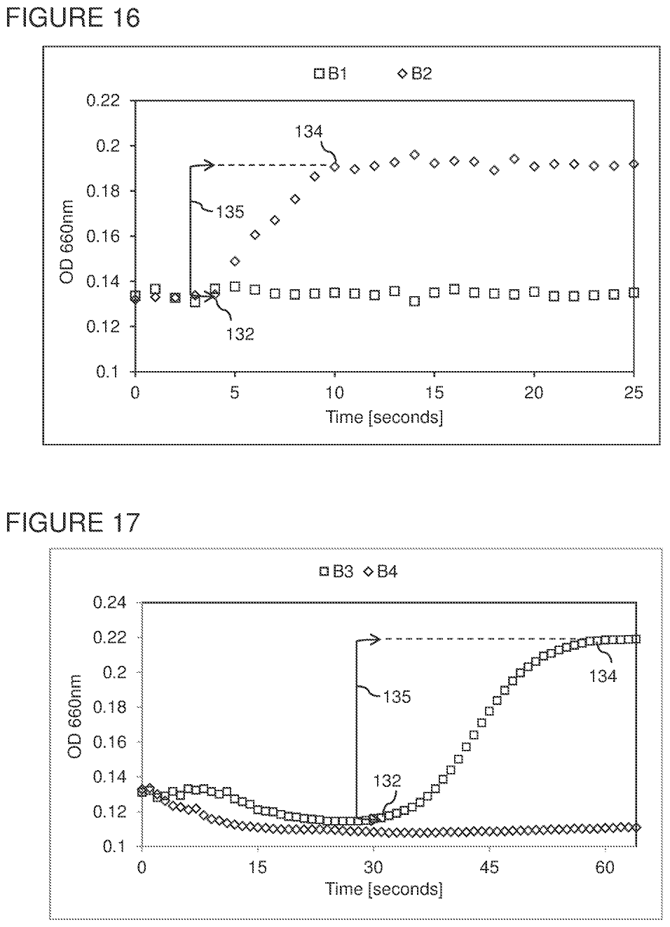

Turning now to FIGS. 16-24 which graphically illustrate the results of specific bioassays performed according to embodiments of the present invention. Unless specifically stated otherwise, these bioassay results illustrate the optical density readings using a kinetic mode at 660 nm over time (in seconds).

Bioassays Employing Tissue Thromboplastin

The results of different bioassays B1-B20 according to methods of the present invention employing varying matrixes for various microparticle dilutions are graphically illustrated in FIGS. 16-33 and a discussion of each is provided below. Each of these bioassays B1-B20 employed a tissue thromboplastin reagent. Specifically, the selected reagents 80 for these bioassays were tissue thromboplastin and CaCl₂) at the amounts listed in each of the protocols below.

B1-B4 Bioassays with BSA Coated Latex & Plain Microparticles

Turning now to FIG. 16 and FIG. 17 which graphically illustrate the results of four bioassays B1-B4, according to the present invention.

The first bioassay B1 employed a microparticle dilution 11 having microparticles 27 with bovine serum albumin (BSA) surface groups. The microparticles were diluted with 0.02% sodium azide in water by a ratio of 1 to 500, for a total microparticle concentration of 0.016%. The optical density of the dilution 11 was measured post dilution at 660 nm to have an optical density value of 0.08. This bioassay B1 further employed a matrix 40 having 0.17M glycine at pH 10.0, 1.0 M NaCl, and 1% simethicone diluted with water. This bioassay B1 was performed with a normal citrated whole blood sample 60.

In comparison, the second bioassay B2 had an identical protocol as bioassay B20 above, only B2 was performed with a microparticle dilution 11 which had microparticles 20 (without BSA surface groups). The selected reagents 80 for this bioassay were tissue thromboplastin and CaCl₂).

As shown in FIG. 16, for the B1 assay, which was an attempt to use the BSA microparticles known to work according to the prior art discussed above, no consistent change of optical density over time was detected, and thus, no clotting start time, clotting end time, or change in clotting were detected for the B1 assay.

Also as shown in FIG. 16, for the B2 assay, which employed the present inventive concept according to the novel method discussed herein, the clotting can be seen to begin 132 at about 4 seconds having an OD value of 0.1342, and clotting can be seen to be finished 134 at about 10 seconds having an OD value of 0.1907, with a change in absorption 135 over time being a change in optical density value having an OD value difference of 0.0565.

To confirm this discrepancy, these bioassays B1, and B2 were repeated again, this time with abnormal blood samples, and for clarity sake, are designated as third and fourth bioassays B3, and B4, respectively.

As shown in FIG. 17, for the B3 assay, again attempting to use the BSA microparticles known to work according to the prior art discussed above, no consistent change of optical density over time was detected, and thus, no clotting start time, clotting end time, or change in clotting were detected for the B3 assay.

Also shown in FIG. 17, for the B4 assay which employed the present inventive methods, the clotting can be seen to begin 132 at about 31 seconds having an OD value of 0.1164, and clotting can be seen to be finished 134 at about 59 seconds having an OD value of 0.2180, with a change in absorption 135 over time being a change in optical density value having an OD value difference of 0.1016.

The protocols for the bioassays B1-B4 are provided in Table 16 below:

TABLE 4
Protocol for Bioassays B1-B4

| Volume | Component |
|---|---|
| 390 uL | Diluted particles |
| 100 uL | Selected Matrix |
| 5 uL | Sample |
| 70 uL | Reagent |

Table 5 below shows the data of the optical density results using a kinetic mode at 660 nm over a period of time for the above described bioassays B1-B4 according to the present invention:

TABLE 5
Optical Density over Time

| Seconds | B1 | B2 | B3 | B4 |
|---|---|---|---|---|
| 0 | 0.1336 | 0.132 | 0.1328 | 0.1311 |
| 1 | 0.1366 | 0.1331 | 0.1334 | 0.132 |
| 2 | 0.1327 | 0.1327 | 0.13 | 0.128 |
| 3 | 0.1308 | 0.1338 | 0.1261 | 0.129 |
| 4 | 0.1367 | 0.1342 | 0.1234 | 0.1316 |
| 5 | 0.1377 | 0.1489 | 0.1226 | 0.1293 |
| 6 | 0.1363 | 0.1605 | 0.1211 | 0.1331 |
| 7 | 0.1346 | 0.1671 | 0.1218 | 0.1323 |
| 8 | 0.1342 | 0.1764 | 0.118 | 0.1331 |
| 9 | 0.1346 | 0.1865 | 0.1156 | 0.1315 |
| 10 | 0.135 | 0.1907 | 0.1149 | 0.13 |
| 11 | 0.1346 | 0.1896 | 0.1133 | 0.1315 |
| 12 | 0.1339 | 0.1911 | 0.1126 | 0.1273 |
| 13 | 0.1357 | 0.1927 | 0.1118 | 0.1257 |
| 14 | 0.1312 | 0.1961 | 0.1114 | 0.1242 |
| 15 | 0.135 | 0.1923 | 0.111 | 0.1211 |
| 16 | 0.1365 | 0.1933 | 0.1106 | 0.1203 |
| 17 | 0.135 | 0.193 | 0.1102 | 0.1199 |
| 18 | 0.1346 | 0.1892 | 0.1098 | 0.1184 |
| 19 | 0.1342 | 0.1942 | 0.1098 | 0.1172 |
| 20 | 0.1354 | 0.1908 | 0.1098 | 0.1168 |
| 21 | 0.1334 | 0.1919 | 0.1098 | 0.116 |
| 22 | 0.1334 | 0.1919 | 0.1098 | 0.1156 |
| 23 | 0.1338 | 0.1911 | 0.1098 | 0.1153 |
| 24 | 0.1342 | 0.1911 | 0.1098 | 0.1145 |
| 25 | 0.135 | 0.192 | 0.1095 | 0.1145 |
| 26 | 0.1354 | 0.192 | 0.1091 | 0.1145 |
| 27 | 0.1362 | 0.192 | 0.1087 | 0.1145 |
| 28 | 0.1362 | 0.1921 | 0.1087 | 0.1145 |
| 29 | 0.1365 | 0.1921 | 0.1087 | 0.1149 |
| 30 | 0.1369 | 0.1921 | 0.1083 | 0.116 |
| 31 | 0.1373 | 0.192 | 0.1083 | 0.1164 |
| 32 | 0.1373 | 0.192 | 0.1079 | 0.1176 |
| 33 | 0.1377 | 0.192 | 0.1079 | 0.1191 |
| 34 | 0.1377 | 0.192 | 0.1079 | 0.1207 |
| 35 | 0.1381 | 0.192 | 0.1079 | 0.1226 |
| 36 | 0.1381 | 0.192 | 0.1079 | 0.1253 |
| 37 | 0.1385 | 0.192 | 0.1079 | 0.1288 |
| 38 | 0.1385 | 0.192 | 0.1079 | 0.1331 |
| 39 | 0.1389 | 0.1921 | 0.1083 | 0.1385 |
| 40 | 0.1389 | 0.1921 | 0.1083 | 0.1439 |
| 41 | 0.1393 | 0.1921 | 0.1083 | 0.1501 |
| 42 | 0.1393 | 0.192 | 0.1083 | 0.1571 |
| 43 | 0.1396 | 0.192 | 0.1087 | 0.164 |
| 44 | 0.1396 | 0.192 | 0.1087 | 0.171 |
| 45 | 0.14 | 0.192 | 0.1087 | 0.1776 |
| 46 | 0.1404 | 0.192 | 0.1087 | 0.1838 |
| 47 | 0.1404 | 0.192 | 0.1087 | 0.1896 |
| 48 | 0.1408 | 0.192 | 0.1091 | 0.195 |
| 49 | 0.1408 | 0.1921 | 0.1091 | 0.1996 |
| 50 | 0.1412 | 0.1921 | 0.1091 | 0.2031 |
| 51 | 0.1412 | 0.1921 | 0.1095 | 0.2062 |
| 52 | 0.1416 | 0.192 | 0.1095 | 0.2093 |
| 53 | 0.142 | 0.192 | 0.1098 | 0.2108 |
| 54 | 0.142 | 0.192 | 0.1098 | 0.2128 |
| 55 | 0.142 | 0.192 | 0.1098 | 0.2143 |
| 56 | 0.1423 | 0.192 | 0.1102 | 0.2155 |
| 57 | 0.1427 | 0.192 | 0.1102 | 0.2166 |
| 58 | 0.1427 | 0.192 | 0.1102 | 0.2178 |
| 59 | 0.1431 | 0.192 | 0.1102 | 0.218 |
| 60 | 0.1431 | 0.192 | 0.1106 | 0.2185 |
| 61 | 0.1431 | 0.1921 | 0.1106 | 0.2186 |
| 62 | 0.1435 | 0.1921 | 0.111 | 0.2186 |
| 63 | 0.1439 | 0.1921 | 0.111 | 0.2188 |
| 64 | 0.1439 | 0.192 | 0.111 | 0.219 |
| 65 | 0.1443 | 0.192 | 0.1114 | 0.219 |
| 66 | 0.1443 | 0.192 | 0.1114 | 0.219 |
| 67 | 0.1447 | 0.192 | 0.1118 | 0.219 |
| 68 | 0.1451 | 0.192 | 0.1118 | 0.219 |
| 69 | 0.1451 | 0.192 | 0.1122 | 0.2188 |
| 70 | 0.1454 | 0.192 | 0.1126 | 0.219 |

B5-B10 Bioassays with Polyethylene Glycol Matrices

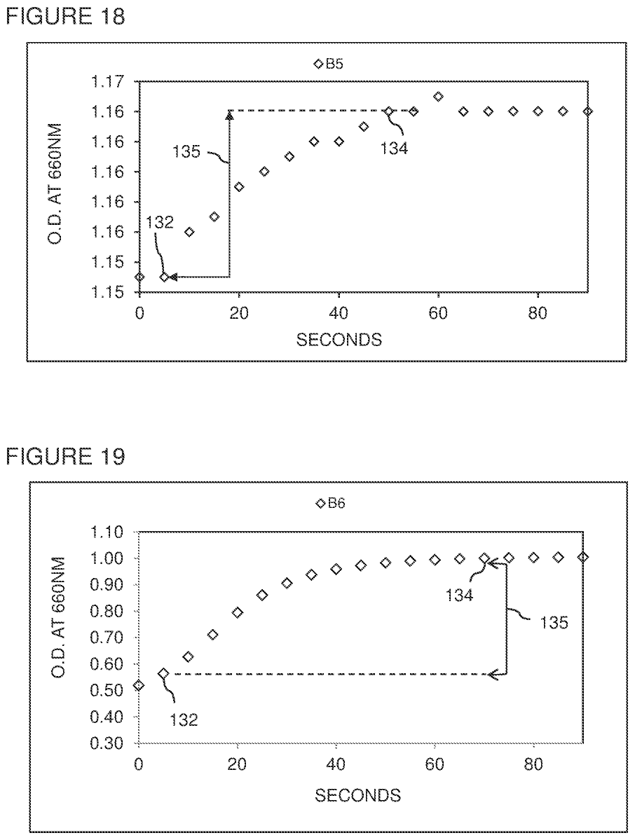

Turning now to FIGS. 18-23 which graphically illustrate the results of six bioassays B5-B10, according to various embodiments of the present invention.

Specifically, FIG. 18 graphically illustrates the results of a first bioassay B5 according to the present invention which employed a microparticle dilution 11 having microparticles 22 with amidine surface groups having a diameter 26 of 95 nm and having a 0.080% weight per volume 28. This bioassay B5 further employed a matrix 40 having 0.17M glycine at pH 7.0, 1.29M NaCl, and 10% Polyethylene glycol (PEG) 20K.

The optical density of the dilution in B5 was measured at 660 nm to have an optical density value 31 of 0.19. As shown in FIG. 18, for the B5 assay, the clotting can be seen to begin 132 at about 5 seconds having an OD value of 1.153, and clotting can be seen to be finished 134 at about 50 seconds having an OD value of 1.164, with a change in absorption 135 over time being a change in optical density value having an OD value difference of 0.011.

FIG. 19 graphically illustrates the results of a second bioassay embodiment B6 which employed a microparticle dilution 11 having microparticles with sulfate surface groups 24 having a diameter 26 of 110 nm and an 0.044 weight per volume 28. This embodiment B6 further employed a matrix 40 having 0.17M glycine at pH 7.0, 1.29M NaCl, and 10% Polyethylene glycol (PEG) 20K.

The optical density of the dilution was measured at 660 nm to have an optical density value of 0.21. As shown in FIG. 19, for the B6 assay, the clotting can be seen to begin 132 at about 5 seconds having an OD value of 0.564, and clotting can be seen to be finished 134 at about 70 seconds having an OD value of 1.000, with a change in absorption 135 over time being a change in optical density value having an OD value difference of 0.436.

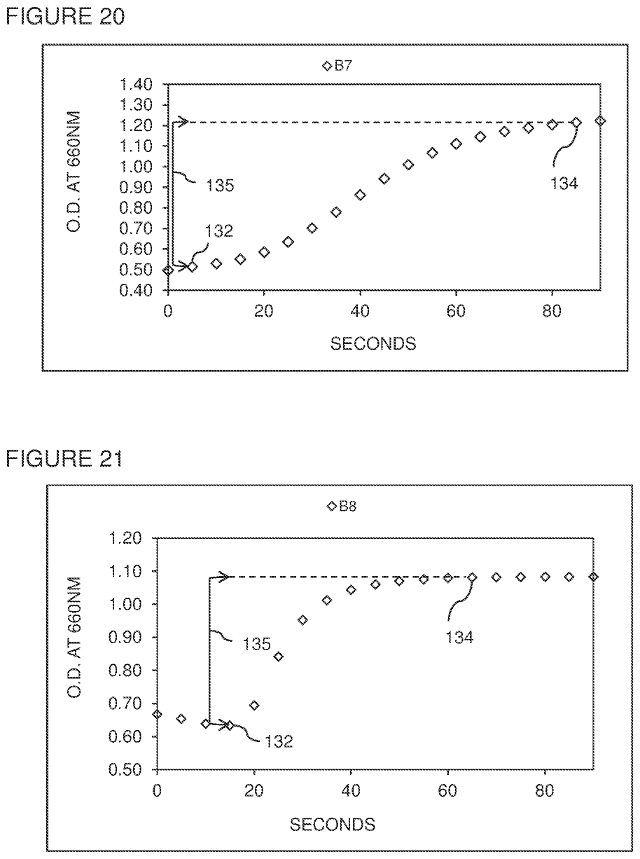

Turning next to FIG. 20, a graphical illustration of the results of a third bioassay embodiment B7 which employed a microparticle dilution 11 having microparticles free of surface groups 20 having a diameter 26 of 96 nm in a 0.067% weight per volume. This dilution having a dilution ratio of 1 to 150 of microparticles to diluent. This embodiment B7 further employed a matrix having 0.17M glycine at pH 7.0, 1.29M NaCl, and 10% Polyethylene glycol (PEG) 20K.

The optical density of the diluted solution was measured post dilution at 660 nm to have an optical density value of 0.21. As shown in FIG. 20, for the B7 assay, the clotting can be seen to begin 132 at about 5 seconds having an OD value of 0.514, and clotting can be seen to be finished 134 at about 85 seconds having an OD value of 1.215, with a change in absorption 135 over time being a change in optical density value having an OD value difference of 0.701.

FIG. 21 then provides a graphical illustration of the results of a fourth bioassay embodiment B8 which employed a microparticle dilution 11 having microparticles with amidine surface groups 22 having a diameter 26 of 95 nm. The microparticle dilution had a 0.080% weight per volume 28. The optical density 31 of the dilution 11 was measured at 660 nm as having an optical density value of 0.19.

This embodiment B8 employed a matrix 40 having 0.17M glycine 47, at pH 7.0, with 1.29M NaCl. The matrix 40 further comprising a carbohydrate derivative in the form of 10% Polyethylene glycol (PEG) 20K and 1% Tween20.

As shown in FIG. 21, for the B8 assay, the clotting can be seen to begin 132 at about 15 seconds having an OD value of 0.633, and clotting can be seen to be finished 134 at about 65 seconds having an OD value of 1.081, with a change in absorption 135 over time being a change in optical density value having an OD value difference of 0.448.

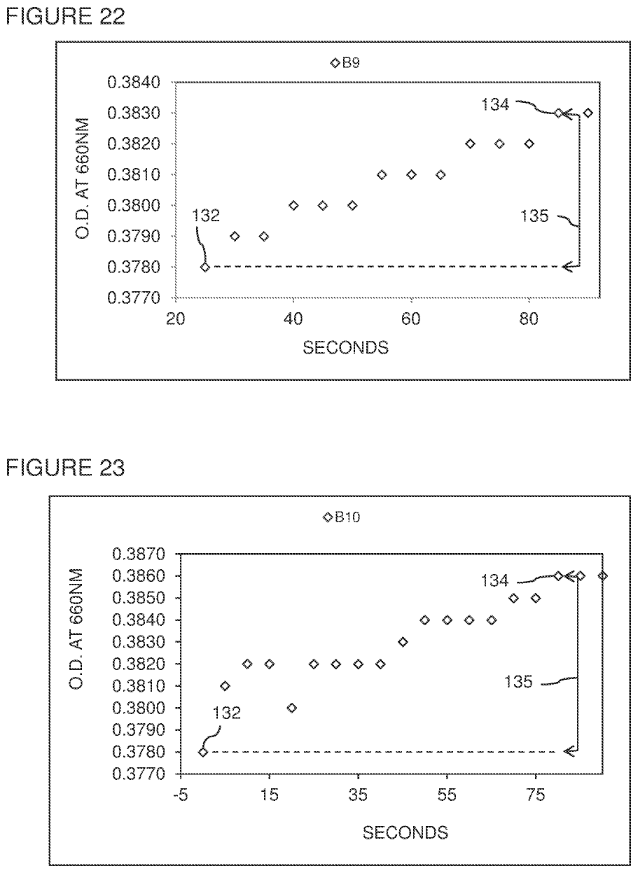

Next, FIG. 22 provides a graphical illustration of the results of a fifth bioassay embodiment B9 which employed a microparticle dilution 11. This dilution 11 has microparticles with sulfate surface groups 24 and a diameter 26 of 110 nm. The microparticles 24 within the dilution 11 were present in an 0.044% weight per volume of the dilution. Specifically, this dilution 11 was diluted with water by a particle to water ratio of 1 to 180. The optical density of the diluted solution was measured post dilution at 660 nm to have an optical density value of 0.21. This embodiment B5 further employed a matrix 40 having 0.17M glycine at pH 7.0, 1.29M NaCl, and 10% Polyethylene glycol (PEG) 20K.

As shown in FIG. 22, for the B9 assay, the clotting can only be detected if the data is analyzed and enhanced as shown (full data is provided in table 7 below). However, when enhanced, the clotting can be seen to begin 132 at about 25 seconds having an OD value of 0.3780, and clotting can be seen to be finished 134 at about 85 seconds having an OD value of 0.3830, with a change in absorption 135 over time being a change in optical density value having an OD value difference of 0.005.

Then, FIG. 23 provides a graphical illustration of the results of a sixth bioassay embodiment B10 which employed a microparticle dilution 11 having microparticles free of surface groups 20 having a diameter of 96 nm in a 0.067% eight per volume. The final dilution had a dilution ratio of microparticles to water of 1 to 150.

The optical density 31 of the dilution 11 was measured post dilution at 660 nm to have an optical density value 31 of 0.21. This embodiment further employed a matrix 40 having 0.17M glycine at pH 7.0, 1.29M NaCl, and 10% Polyethylene glycol (PEG) 20K.

As shown in FIG. 23, for the B10 assay, the clotting can only be detected if the data is analyzed and enhanced as shown (full data is provided in table 7 below). However, when enhanced, the clotting can be seen to begin 132 immediately having an OD value of 0.3780, and clotting can be seen to be finished 134 at about 85 seconds having an OD value of 0.3860, with a change in absorption 135 over time being a change in optical density value having an OD value difference of 0.008.

The protocol for all six embodiments B5-B10 listed above are shown in Table 4 below:

TABLE 6
Bioassays B5-B10 Component Protocol

| Volume | Component |
|---|---|
| 395 uL | Diluted particles |
| 50 uL | Selected Matrix |
| 5 uL | Citrated whole blood |
| 50 uL | Reagent |

The general methods for each of these bioassays B1-B6 were identical and generally prepared as discussed above with reference to FIG. 10.

Table 5 shows the data of the corrected optical density results using a kinetic mode at 660 nm over a minute and a half for each of the above six described bioassay embodiments according to the present invention:

TABLE 7
Optical Density Against Time

| (secs) | B5 | B6 | B7 | B8 | B9 | B10 |
|---|---|---|---|---|---|---|
| 0 | 1.153 | 0.519 | 0.496 | 0.667 | 0.378 | 0.378 |
| 5 | 1.153 | 0.564 | 0.514 | 0.654 | 0.380 | 0.381 |
| 10 | 1.156 | 0.627 | 0.530 | 0.639 | 0.379 | 0.382 |
| 15 | 1.157 | 0.711 | 0.551 | 0.633 | 0.380 | 0.382 |
| 20 | 1.159 | 0.795 | 0.585 | 0.694 | 0.379 | 0.380 |
| 25 | 1.160 | 0.861 | 0.635 | 0.842 | 0.378 | 0.382 |
| 30 | 1.161 | 0.906 | 0.702 | 0.952 | 0.379 | 0.382 |
| 35 | 1.162 | 0.938 | 0.780 | 1.012 | 0.379 | 0.382 |
| 40 | 1.162 | 0.959 | 0.863 | 1.043 | 0.380 | 0.382 |
| 45 | 1.163 | 0.973 | 0.942 | 1.060 | 0.380 | 0.383 |
| 50 | 1.164 | 0.983 | 1.010 | 1.070 | 0.380 | 0.384 |
| 55 | 1.164 | 0.990 | 1.067 | 1.075 | 0.381 | 0.384 |
| 60 | 1.165 | 0.994 | 1.111 | 1.079 | 0.381 | 0.384 |
| 65 | 1.164 | 0.998 | 1.145 | 1.081 | 0.381 | 0.384 |
| 70 | 1.164 | 1.000 | 1.171 | 1.082 | 0.382 | 0.385 |
| 75 | 1.164 | 1.002 | 1.189 | 1.082 | 0.382 | 0.385 |
| 80 | 1.164 | 1.003 | 1.204 | 1.083 | 0.382 | 0.386 |
| 85 | 1.164 | 1.004 | 1.215 | 1.083 | 0.383 | 0.386 |
| 90 | 1.164 | 1.005 | 1.223 | 1.083 | 0.383 | 0.386 |

B11-B12 Bioassays with Simethicone Matrices

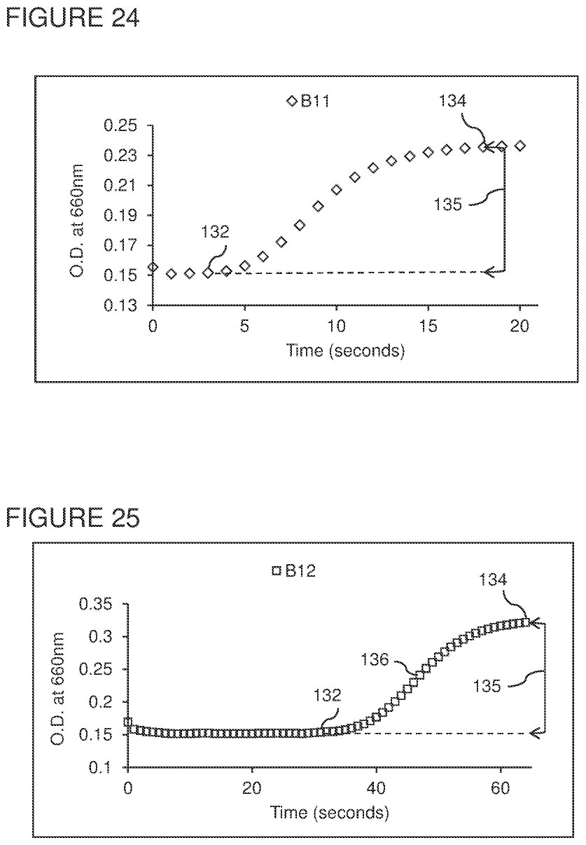

Turning now to FIG. 24 which graphically illustrates the results of a first bioassay B11 according to the present invention which employs a microparticle dilution 11 having microparticles 24 with sulfate surface groups having a diameter of 100 nm. The microparticles are diluted with 0.02% sodium azide in water by a ratio of 1 to 500, for a total microparticle concentration of 0.016%.

The optical density of the dilution 11 was measured post dilution at 660 nm to have an optical density value of 0.08. This bioassay B11 further employed a matrix 40 having 0.17M glycine at pH 10.0, 1.29M NaCl, and 1% simethicone diluted with water for injection grade purified water to a concentration supporting less than 15 second de-foaming performance in a USP Assay under the commercial identity of FoamAWAY™. This bioassay B11 was performed with a normal sample 60.

As shown in FIG. 24, for the B11 assay, the clotting can be seen to begin 132 at about 3 seconds having an OD value of 0.1516, and clotting can be seen to be finished 134 at about 65 seconds having an OD value of 0.2356, with a change in absorption 135 over time being a change in optical density value having an OD value difference of 0.084.

In comparison, FIG. 25 graphically illustrates the results of bioassay B12 having an identical protocol as bioassay B11 above, only now performed with an abnormal sample. As shown in FIG. 25, for the B12 assay, the clotting can now be seen to begin 132 at about 30 seconds having an OD value of 0.1528, and clotting can be seen to be finished 134 at about 65 seconds having an OD value of 0.3215, with a change in absorption 135 over time being a change in optical density value having an OD value difference of 0.1687.

The protocols for the bioassays B11, B12 are provided in Table 16 below:

TABLE 8
Protocol for Bioassays B11, B12

| Volume | Component |
|---|---|
| 390 uL | Diluted particles |
| 100 uL | Selected Matrix |
| 5 uL | Sample |
| 70 uL | Reagent |

Table 9 below shows the data of the optical density results using a kinetic mode at 660 nm for the above described bioassays B11-B12 according to the present invention, over a minute for bioassay B12, and over 20 seconds for B11:

TABLE 9
Optical Density over Time

| Time (Seconds) | B11 | B12 |
|---|---|---|
| 0 | 0.1555 | 0.1691 |
| 1 | 0.1509 | 0.1582 |
| 2 | 0.1513 | 0.1563 |
| 3 | 0.1516 | 0.1547 |
| 4 | 0.1528 | 0.154 |
| 5 | 0.1563 | 0.1532 |
| 6 | 0.1625 | 0.152 |
| 7 | 0.1722 | 0.1516 |
| 8 | 0.1834 | 0.1513 |
| 9 | 0.1961 | 0.1516 |
| 10 | 0.207 | 0.1516 |
| 11 | 0.2155 | 0.152 |
| 12 | 0.2217 | 0.1524 |
| 13 | 0.2263 | 0.1524 |
| 14 | 0.2294 | 0.1516 |
| 15 | 0.2321 | 0.1516 |
| 16 | 0.2337 | 0.1516 |
| 17 | 0.2349 | 0.1516 |
| 18 | 0.2356 | 0.1516 |
| 19 | 0.236 | 0.1516 |
| 20 | 0.2364 | 0.1516 |
| 21 | | 0.152 |
| 22 | | 0.152 |
| 23 | | 0.152 |
| 24 | | 0.1524 |
| 25 | | 0.152 |
| 26 | | 0.152 |
| 27 | | 0.152 |
| 28 | | 0.1516 |
| 29 | | 0.1524 |
| 30 | | 0.1528 |
| 31 | | 0.1536 |
| 32 | | 0.1544 |
| 33 | | 0.1547 |
| 34 | | 0.1559 |
| 35 | | 0.1574 |
| 36 | | 0.1598 |
| 37 | | 0.1629 |
| 38 | | 0.1663 |
| 39 | | 0.171 |
| 40 | | 0.1768 |
| 41 | | 0.1838 |
| 42 | | 0.1915 |
| 43 | | 0.2004 |
| 44 | | 0.2097 |
| 45 | | 0.2198 |
| 46 | | 0.2298 |
| 47 | | 0.241 |
| 48 | | 0.2511 |
| 49 | | 0.2604 |
| 50 | | 0.2689 |
| 51 | | 0.2767 |
| 52 | | 0.284 |
| 53 | | 0.2906 |
| 54 | | 0.296 |
| 55 | | 0.3006 |
| 56 | | 0.3049 |
| 57 | | 0.3088 |
| 58 | | 0.3115 |
| 59 | | 0.3142 |
| 60 | | 0.3161 |
| 61 | | 0.3177 |
| 62 | | 0.3192 |
| 63 | | 0.3204 |
| 64 | | 0.3215 |

It is to be understood that while these optical density values are being shown here, these values are not normally presented to the user. Instead, according to the method discussed above, the clotting time is corrected using the hemoglobin measurement for the adjusted true plasma value of the sample. This corrected clotting time is then used to report a normalized INR as discussed above.

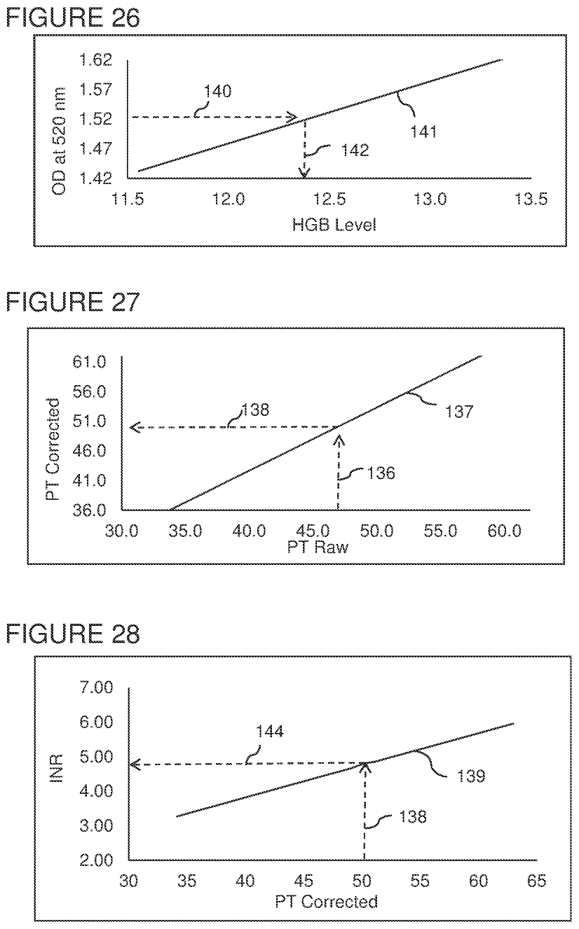

For example, for the abnormal blood bioassay B12 above, a first optical density measurement 116′ was taken in order to determine the hemoglobin content. The optical density detected at 530 nm was 1.5135, and this optical density value 140 was compared with a predetermined relationship 141 of OD values to known hemoglobin values in order to determine the specific hemoglobin level 142 present in the sample being analyzed. Again, although this level is not normally reported to the client or user, this analysis is graphically illustrated in FIG. 26. This specific HGB level 142 for the bioassay B11 was then stored in system memory until after the second set of optical density measurements 116 were finished.

As discussed above with reference to FIG. 25, for the B12 assay at this hemoglobin level, the clotting started 132 at a first time (30 seconds) having an OD value of 0.1528, and the clotting ended 134 at a second time (65 seconds) having an OD value of 0.3215. The change in absorption 135 over a delta time (35 seconds) was a first OD value difference of 0.1687. Change in absorption 135 over a change in time resulting in OD value differences for various bioassays of the present inventive method range between 0.005-1.0. More preferably, the OD value difference is at least 0.2, and when necessary, the OD value difference is at least 0.08. This change in absorption 135 can be used to calculate a PT value 136 (47 seconds which was associated with the absorption OD value of 0.23715).

The system then uses this initial PT value 136 and retrieves the specific HGB level 142 as determined with reference to FIG. 24. A predetermined relationship 137 is then used to determine an adjusted or corrected PT value 138. Specifically, FIG. 27 illustrates the correction of the uncorrected PT value 136 through a predetermined relationship 137 corresponding with the hemoglobin level 142 (12.3) of the sample 60. This relationship 137 is then used to provide a corrected PT value 138. Where for the hemoglobin level 142, the relationship 137 is mathematically expressed as:

CPT=PT*SQRT(C/HGB)

Where CPT is the corrected prothrombin time 138; PT is the uncorrected prothrombin time 136; C is a hemoglobin constant; and HGB is the specific hemoglobin level 142 associated with a specific sample 60.

FIG. 28 then illustrates the calculation of the INR from the corrected PT 138. Specifically, this calculation is:

INR=(PT/MT)^{ISI}

Where for the above formula applied to data obtained for B12, PT stands for the prothrombin corrected test time 138 (50.1 seconds); ISI stands for the sensitivity index of the thromboplastin used (0.98 no associated units); MT stands for the mean prothrombin time derived from 20 normal samples (10.2 seconds); and INR stands for the normalization value 144 (4.8 no associated units). In general practice, only the corrected PT value (50.1 seconds) and INR value (4.8 no associated units) will be reported to the user.

B13-B14 Bioassays with Carboxyl Microparticle

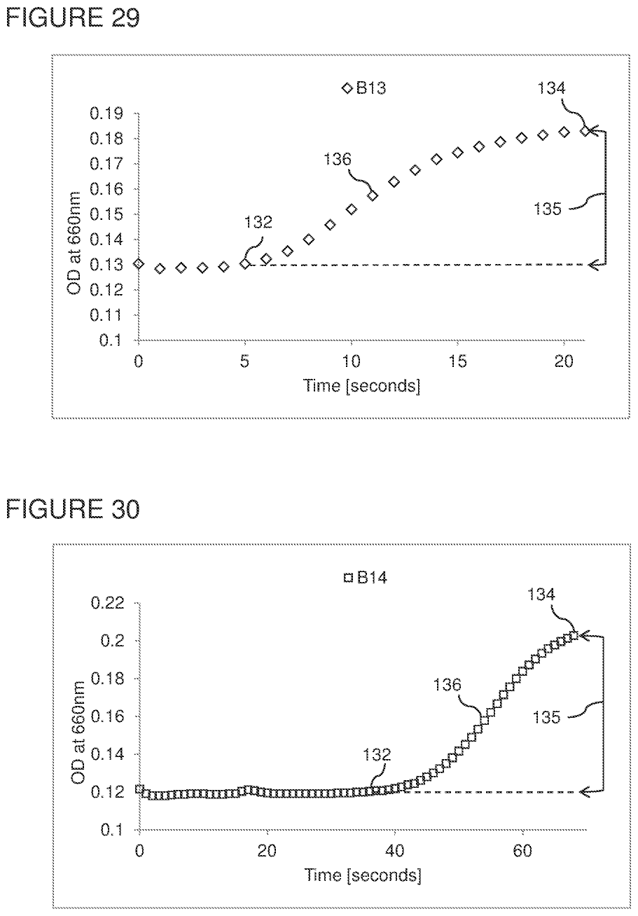

Turning now to FIG. 29 which graphically illustrates the results of a bioassay B13 according to the present invention which employed a microparticle dilution 11 having microparticles 25 with carboxyl surface groups having a diameter of 103 nm in a 0.016% weight per volume dilution. The present bioassay methods using carboxyl microparticles 25 call for a dilution 11 having a dilution ratio 30 of microparticles 25 to dihydrogen monoxide 33 in ranges of 1:50 to 1:2000; and more preferably in a range of 1:100 to 1:1000.

The optical density of the diluted solution was measured post dilution at 660 nm to have an optical density value of 0.08. This bioassay B13 further employed a matrix having 0.17M glycine at pH 10.0, 1M NaCl, and 1% simethicone diluted with water for injection grade purified water to a concentration supporting less than 15 second de-foaming performance in a USP Assay under the commercial identity of FoamAWAY™. This bioassay B13 was performed with a normal sample 60.

As shown in FIG. 29, for the B13 assay, the clotting can be seen to begin 132 at about 5 seconds having an OD value of 0.1304, and clotting can be seen to be finished 134 at about 20 seconds having an OD value of 0.1826, with a change in absorption 135 over time being a change in optical density value having an OD value difference of 0.0522.

In comparison, FIG. 30 graphically illustrates the results of bioassay B14 having an identical protocol as bioassay B13 above, only now performed with an abnormal sample. As shown in FIG. 30, for the B14 assay, the clotting can be seen to begin 132 at about 40 seconds having an OD value of 0.1218, and clotting can be seen to be finished 134 at about 68 seconds having an OD value of 0.2027, with a change in absorption 135 over time being a change in optical density value having an OD value difference of 0.0811.

The protocols for the bioassays B13, B14 are provided in Table 10 below.

TABLE 10
Protocol for Bioassays B13, B14

| Volume | Component |
|---|---|
| 390 uL | Diluted particles |
| 100 uL | Selected Matrix |
| 5 uL | Sample |
| 70 uL | Reagent |

Table 11 below shows the data of the optical density results using a kinetic mode at 660 nm for the above described bioassays B13-B14 according to the present invention, over a minute for bioassay B13, and over 20 seconds for B14:

TABLE 11
Optical Density over Time

| Time (Seconds) | B13 | B14 |
|---|---|---|
| 0 | 0.1304 | 0.1215 |
| 1 | 0.1284 | 0.1191 |
| 2 | 0.1288 | 0.118 |
| 3 | 0.1288 | 0.118 |
| 4 | 0.1292 | 0.118 |
| 5 | 0.1304 | 0.1184 |
| 6 | 0.1323 | 0.1187 |
| 7 | 0.1354 | 0.1187 |
| 8 | 0.14 | 0.1191 |
| 9 | 0.1458 | 0.1191 |
| 10 | 0.152 | 0.1191 |
| 11 | 0.1574 | 0.1187 |
| 12 | 0.1629 | 0.1187 |
| 13 | 0.1675 | 0.1187 |
| 14 | 0.1718 | 0.1191 |
| 15 | 0.1745 | 0.1191 |
| 16 | 0.1768 | 0.1203 |
| 17 | 0.1787 | 0.1211 |
| 18 | 0.1803 | 0.1207 |
| 19 | 0.1814 | 0.1199 |
| 20 | 0.1826 | 0.1195 |
| 21 | 0.183 | 0.1191 |
| 22 | | 0.1191 |
| 23 | | 0.1191 |
| 24 | | 0.1191 |
| 25 | | 0.1191 |
| 26 | | 0.1191 |
| 27 | | 0.1191 |
| 28 | | 0.1191 |
| 29 | | 0.1191 |
| 30 | | 0.1191 |
| 31 | | 0.1195 |
| 32 | | 0.1195 |
| 33 | | 0.1195 |
| 34 | | 0.1199 |
| 35 | | 0.1199 |
| 36 | | 0.1203 |
| 37 | | 0.1207 |
| 38 | | 0.1207 |
| 39 | | 0.1211 |
| 40 | | 0.1218 |
| 41 | | 0.1226 |
| 42 | | 0.1238 |
| 43 | | 0.1245 |
| 44 | | 0.1261 |
| 45 | | 0.128 |
| 46 | | 0.13 |
| 47 | | 0.1323 |
| 48 | | 0.135 |
| 49 | | 0.1381 |
| 50 | | 0.1416 |
| 51 | | 0.1451 |
| 52 | | 0.1489 |
| 53 | | 0.1532 |
| 54 | | 0.1578 |
| 55 | | 0.1621 |
| 56 | | 0.1667 |
| 57 | | 0.1714 |
| 58 | | 0.1756 |
| 59 | | 0.1799 |
| 60 | | 0.1838 |
| 61 | | 0.1872 |
| 62 | | 0.1903 |
| 63 | | 0.1934 |
| 64 | | 0.1958 |
| 65 | | 0.1977 |
| 66 | | 0.1996 |
| 67 | | 0.2012 |
| 68 | | 0.2027 |

B15-B18 Bioassays with Differing Operating Temperatures

Typically, many bioassays according to the present invention are performed at physiologic temperatures, but this is not always the case. It is to be understood that in general, most temperatures can be applied to methods of the present invention. However, adjusting this variable with the bioassays discussed above will introduce potential variations such that the bioassay results will generally be altered. Thus, temperature variations should not be introduced without accounting for these variations, especially in clotting assays where accurate timing can impact clinical outcomes.

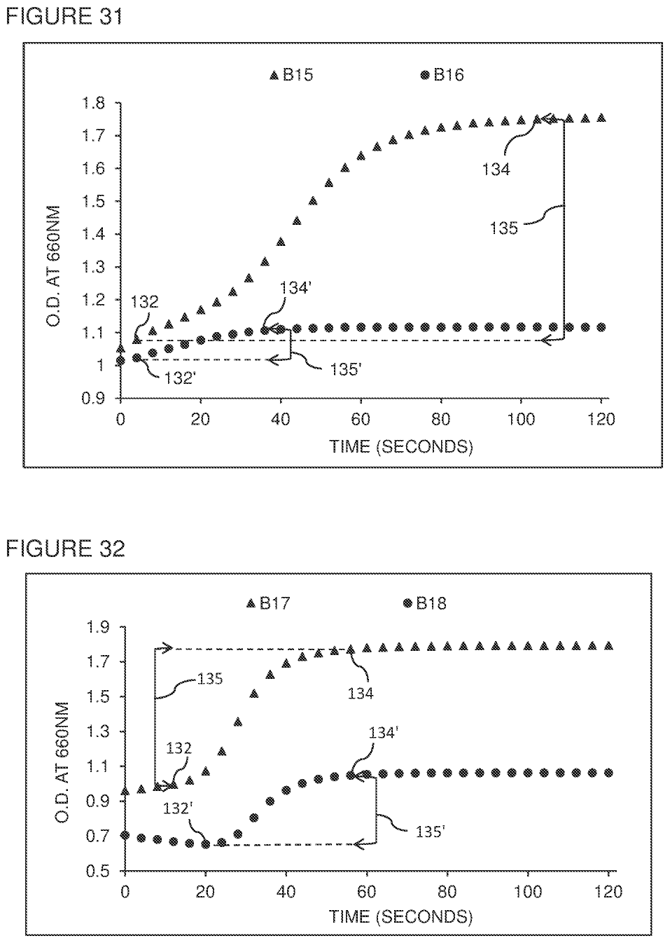

FIGS. 31 and 32 graphically illustrate the results of four bioassay embodiments B15-B18 of the present invention employing two different microparticle matrixes at two different temperatures. The protocols for all four bioassay embodiments are provided in Table 12 below:

TABLE 12
Protocol for Bioassays 15-18

| Volume | Component |
|---|---|
| 395 uL | Diluted particles |
| 50 uL | Selected Matrix |
| 5 uL | Citrated whole blood |
| 50 uL | Selected Reagent |

Specifically, the bioassay B15 employed a microparticle dilution 11 having microparticles with amidine surface groups 22 having a diameter of 95 nm. At 0.080% weight per volume, this dilution 11 had microparticles 22 diluted with water by a ratio of 1 to 50. The optical density of the diluted solution was measured post dilution at 660 nm to have an optical density value of 0.19. This bioassay B15 employed a matrix 40 having 0.17M glycine at pH 7.0, 0.29M NaCl, and 10% Polyethylene glycol (PEG) 20K. This bioassay B15 was performed at a temperature of 22 C.

As shown in FIG. 31, for the B15 assay, the clotting can be seen to begin 132 almost immediately, at about 4 seconds having an OD value of 1.079, and clotting can be seen to be finished 134 at about 100 seconds having an OD value of 1.748, with a change in absorption 135 over time being a change in optical density value having an OD value difference of 0.669.

Also seen in FIG. 31 is a graphical representation of the results of a similar bioassay B16. This bioassay B16 also employed a microparticle dilution 11 having microparticles 22 with amidine surface groups having a diameter of 95 nm. Present in a 0.080% weight per volume, the microparticles 22 were diluted with water by a dilution ratio 30 of 1 to 50. The optical density 31 of the dilution was measured post dilution at 660 nm to have an optical density value 31 of 0.19. This bioassay B16 also employed a matrix 40 having 0.17M glycine at pH 7.0, 0.29M NaCl, and 10% Polyethylene glycol (PEG) 20K. However, unlike bioassay B15, this bioassay B16 was performed at an operating temperature of 37 C.

As also shown in FIG. 31, for the B16 assay, the clotting values are harder to distinguish, though clotting can be seen to begin 132′ again almost immediately, at about 4 seconds having an OD value of 1.023. Unlike the B15 assay, the B16 assay appears to finish clotting quickly, as clotting appears to be finished 134′ at about 40 seconds having an OD value of 1.110, with a change in absorption 135′ over time being a change in optical density value having an OD value difference of only 0.087.

Turning next to FIG. 32, there is a graphical representation of the results of another pair of bioassays B17 and B18 according to the present invention. In the first bioassay B17, the microparticle dilution 11 employed microparticles 22 with amidine surface groups having a diameter 26 of 0.95 nm. Diluted with water by a ratio of 1 to 50, the microparticles 22 in this bioassay B17 were present in the dilution 11 at 0.080% weight per volume.

The optical density 31 of the dilution 11 was measured at 660 nm to have an optical density value of 0.19. This bioassay B17 employed a matrix 40 having 0.17M glycine at pH 7.0, 0.29M NaCl, 10% Polyethylene glycol (PEG) 20K, and 1% Tween20. The bioassay B17 was performed at an operating temperature of 22 C.

As shown in FIG. 32, for the B17 assay, the clotting can be seen to begin 132 at about 12 seconds having an OD value of 0.997, and clotting can be seen to be finished 134 at about 58 seconds having an OD value of 1.772, with a change in absorption 135 over time being a change in optical density value having an OD value difference of 0.775.

The second bioassay B18 according to the present invention, used a microparticle dilution which employed microparticles 22 with amidine surface groups having a diameter of 0.95 nm in a 0.080% weight per volume. The dilution 11 had a dilution ratio of 1 to 50. The optical density of the diluted solution was measured post dilution at 660 nm to have an optical density value of 0.19. This bioassay B18 also employed a matrix having 0.17M glycine at pH 7.0, 0.29M NaCl, 10% Polyethylene glycol (PEG) 20K, and 1% Tween20. However, unlike bioassay B17, the bioassay B18 was performed at an operating temperature of 37 C.

As also shown in FIG. 32, for the B18 assay, the clotting values are harder to distinguish, though clotting can be seen to begin 132′ again at about 20 seconds having an OD value of 0.653. The B18 assay appears to finish clotting 134′ at about 60 seconds having an OD value of 1.054, with a change in absorption 135′ over time being a change in optical density value having an OD value difference of only 0.401.

The general methods for each of these bioassays B15-B18 were identical and generally prepared as discussed above with reference to FIG. 10.

Table 13 below shows the data of the corrected optical density results using a kinetic mode at 660 nm over minutes for each of the above described bioassays B15-B18 according to the present invention:

TABLE 13
Optical Density over Time

| TIME | B15 | B16 | B17 | B18 |
|---|---|---|---|---|
| 0 | 1.053 | 1.015 | 0.961 | 0.705 |
| 4 | 1.079 | 1.023 | 0.971 | 0.688 |
| 8 | 1.105 | 1.038 | 0.984 | 0.68 |
| 12 | 1.126 | 1.051 | 0.997 | 0.668 |
| 16 | 1.147 | 1.064 | 1.02 | 0.658 |
| 20 | 1.169 | 1.077 | 1.073 | 0.653 |
| 24 | 1.193 | 1.088 | 1.187 | 0.663 |
| 28 | 1.225 | 1.095 | 1.356 | 0.711 |
| 32 | 1.266 | 1.102 | 1.518 | 0.805 |
| 36 | 1.317 | 1.106 | 1.628 | 0.899 |
| 40 | 1.377 | 1.11 | 1.692 | 0.963 |
| 44 | 1.441 | 1.112 | 1.729 | 1.002 |
| 48 | 1.502 | 1.113 | 1.75 | 1.026 |
| 52 | 1.557 | 1.114 | 1.764 | 1.04 |
| 56 | 1.603 | 1.116 | 1.772 | 1.048 |
| 60 | 1.639 | 1.116 | 1.779 | 1.054 |
| 64 | 1.667 | 1.116 | 1.782 | 1.057 |
| 68 | 1.687 | 1.116 | 1.785 | 1.059 |
| 72 | 1.703 | 1.116 | 1.787 | 1.061 |
| 76 | 1.716 | 1.116 | 1.788 | 1.062 |
| 80 | 1.725 | 1.116 | 1.79 | 1.062 |
| 84 | 1.731 | 1.116 | 1.791 | 1.062 |
| 88 | 1.738 | 1.117 | 1.791 | 1.063 |
| 92 | 1.741 | 1.117 | 1.792 | 1.063 |
| 96 | 1.745 | 1.117 | 1.791 | 1.063 |
| 100 | 1.748 | 1.117 | 1.792 | 1.063 |
| 104 | 1.75 | 1.117 | 1.792 | 1.063 |
| 108 | 1.752 | 1.117 | 1.792 | 1.063 |
| 112 | 1.753 | 1.116 | 1.792 | 1.063 |
| 116 | 1.753 | 1.116 | 1.793 | 1.063 |
| 120 | 1.755 | 1.116 | 1.793 | 1.063 |

B19-B20 Bioassays for Monitoring Anticoagulant Use

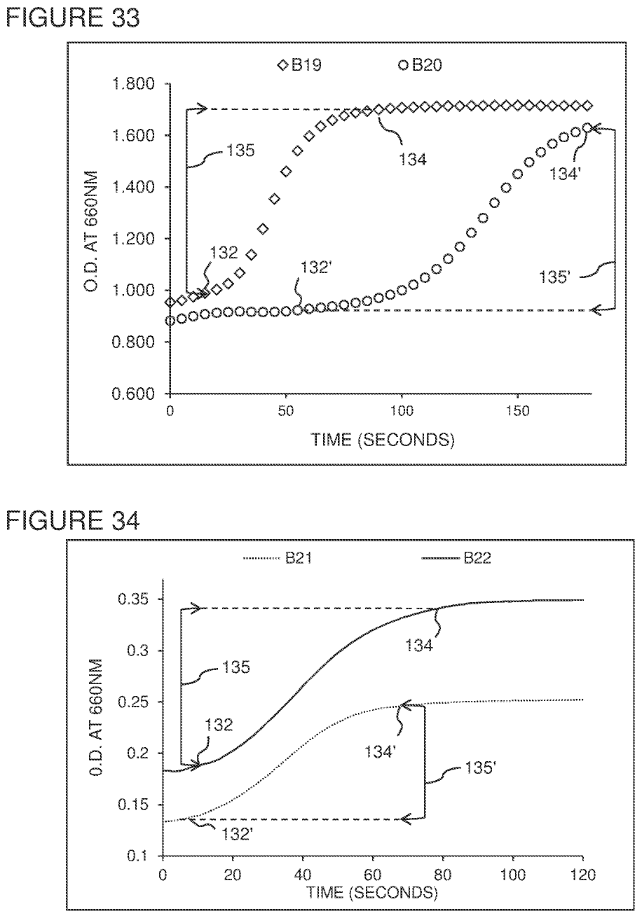

Turning now to FIG. 33 which graphically illustrates the results of prothrombin time bioassays B19, B20 which measure the extrinsic pathway of coagulation and monitors oral anticoagulant use, multistep. These bioassays B11, B12 are prothrombin time assays illustrating the results of measurements of optical density versus time in seconds for a first assay B19 of the present inventive method of a normal whole blood sample vs. a second assay B20 of the present inventive method of a sample from a patient taking the oral anticoagulant Coumadin™.

Coumadin™ (also known as warfarin) inhibits Vitamin K synthesis and therefore inhibits the half-life of Factor VII. Factor VII levels are assayed and corrected in this graph using the prothrombin time test and dual wavelength correction as described herein.

As illustrated in FIG. 33, normal clot times 132 are typically seen beginning after about 15 to 20 seconds where clot formation is initiated via the extrinsic coagulation pathway via Factor VII activation as discussed above. Specifically, as illustrated in FIG. 33, the assay B19 for normal blood sample begins clotting 132 at approximately 15 seconds and the assay B20 for the Coumadin sample begins clotting 132′ at approximately 100 seconds.

For both of these assays, B19 and B20, a microparticle dilution 11 employed microparticles 22 with amidine surface groups having a diameter 26 of 95 nm in a 0.080% weight per volume. These microparticle dilutions 11 were diluted with water by a ratio of 1 to 50. The optical density of the dilutions were measured post dilution at 660 nm to have an optical density value of 0.19.

As shown in FIG. 33, for the B19 assay, the clotting can be seen to begin 132 at about 15 seconds having an OD value of 0.989, and clotting can be seen to be finished 134 at about 90 seconds having an OD value of 1.700, with a change in absorption 135 over time being a change in optical density value having an OD value difference of 0.711.

Also shown in FIG. 33, for the B20 assay, the clotting can be seen to begin 132′ at about 50 seconds having an OD value of 0.919, and clotting can be seen to be finished 134′ at about 180 seconds having an OD value of 1.6290, with a change in absorption 135′ over time being a change in optical density value having an OD value difference of 0.7100.

The protocol for the bioassays B19, B20 is provided in Table 14 below:

TABLE 14
Protocol for Bioassays 19, 20

| Volume | Component |
|---|---|
| 345 uL | Diluted particles |
| 50 uL | Selected Matrix |
| 5 uL | Sample |
| 50 uL | Tissue Thromboplastin (Innovin) w/CaCl2 |

The general methods for each of these bioassays B19-B20 were identical and generally prepared as discussed above with reference to FIG. 10. Table 15 shows the data of the optical density using a kinetic mode at 660 nm over two minutes for the first bioassay B19 with normal citrated blood and for the second bioassay B20 with coumadin citrated blood:

TABLE 15
Optical Density over Time

| TIME (secs) | B19 | B20 |
|---|---|---|
| 0 | 0.954 | 0.883 |
| 5 | 0.961 | 0.890 |
| 10 | 0.975 | 0.900 |
| 15 | 0.989 | 0.908 |
| 20 | 1.003 | 0.913 |
| 25 | 1.026 | 0.916 |
| 30 | 1.067 | 0.918 |
| 35 | 1.138 | 0.917 |
| 40 | 1.238 | 0.916 |
| 45 | 1.354 | 0.917 |
| 50 | 1.460 | 0.919 |
| 55 | 1.540 | 0.923 |
| 60 | 1.597 | 0.928 |
| 65 | 1.635 | 0.933 |
| 70 | 1.659 | 0.938 |
| 75 | 1.676 | 0.944 |
| 80 | 1.687 | 0.951 |
| 85 | 1.694 | 0.959 |
| 90 | 1.700 | 0.970 |
| 95 | 1.704 | 0.983 |
| 100 | 1.707 | 1.000 |
| 105 | 1.709 | 1.022 |
| 110 | 1.711 | 1.049 |
| 115 | 1.712 | 1.082 |
| 120 | 1.714 | 1.122 |
| 125 | 1.714 | 1.169 |
| 130 | 1.715 | 1.223 |
| 135 | 1.715 | 1.280 |
| 140 | 1.716 | 1.339 |
| 145 | 1.716 | 1.397 |
| 150 | 1.716 | 1.450 |
| 155 | 1.716 | 1.496 |
| 160 | 1.715 | 1.535 |
| 165 | 1.715 | 1.567 |
| 170 | 1.715 | 1.593 |
| 175 | 1.715 | 1.613 |
| 180 | 1.715 | 1.629 |

Bioassays Employing Thrombin

The results of different bioassays B21-B22 according to methods of the present invention employing varying matrixes for various microparticle dilutions are graphically illustrated in FIG. 34 and a discussion of each is provided below. Each of these bioassays B21-B22 employed a thrombin reagent. Specifically, the selected reagents 80 for these bioassays were tissue thromboplastin and CaCl₂) at the amounts listed in each of the protocols below.

B21-B22 Bioassays for Measuring Fibrinogen Levels

Turning now to FIG. 34 which graphically illustrates the results of Thrombin Time (TT) Assays B21, B22 which directly measures the fibrinogen level and function and also will determine if thrombin inhibitors are present in the sample. FIG. 34 illustrates the results of measurements of optical density versus time in seconds for a first TT assay B21 of the present inventive method with 30 uL Thrombin reagent for citrated plasma versus a second TT assay B22 with 20 uL Thrombin reagent and citrated blood according to the present inventive method.

The protocols for the bioassays B15, B16 are provided in Table 16 below:

TABLE 16
Protocol for Bioassays 21, 22

| Volume | Component |
|---|---|
| 390 uL | Diluted particles |
| 70 uL | Selected Matrix |
| 5 uL | Sample (Plasma or Blood) |
| 20 uL or 30 uL | Thrombin time (TT) reagent |

Specifically, these bioassays B21, B22 employed a microparticle dilution 11 having microparticles 24 with sulfate surface groups having a diameter 26 of 110 nm in an 0.2% weight per volume. Both bioassays B21, B22 employed a matrix 40 having 0.17M glycine at pH 10.0, 1.0M NaCl. The first bioassay B21 employed 30 uL of Siemens™ thrombin time (TT) reagent, and the second bioassay B22 employed 20 uL of Siemens™ thrombin time (TT) reagent.

Some thrombin inhibitors which may be used with the present inventive method are unfractionated heparins, low molecular weight heparins, and direct anti-thrombin oral anticoagulants including but not limited to hirudin, rivaroxaban, apixaban, dabigatran and argatroban. The general methods for each of these bioassays B21-B22 were identical and generally prepared as discussed above with reference to FIG. 10.

As shown in FIG. 34, for the B21 assay, the clotting can be seen to begin 132 at about 5 seconds having an OD value of 0.1829, and clotting can be seen to be finished 134 at about 100 seconds having an OD value of 0.3481, with a change in absorption 135 over time being a change in optical density value having an OD value difference of 0.1652.

Also shown in FIG. 34, for the B22 assay, the clotting can be seen to begin 132′ at about 5 seconds having an OD value of 0.1357, and clotting can be seen to be finished 134′ at about 80 seconds having an OD value of 0.2491, with a change in absorption 135′ over time being a change in optical density value having an OD value difference of 0.1134.

Table 17 shows the data of the optical density using a kinetic mode at 660 nm over three minutes for the first bioassay B21 with 30 uL Thrombin Reagent and for the second bioassay B22 with 20 uL Thrombin Reagent.

TABLE 17
Optical Density over Time

| Time (Secs) | B21 | B22 |
|---|---|---|
| 0 | 0.1334 | 0.1829 |
| 4 | 0.1349 | 0.1825 |
| 8 | 0.138 | 0.1863 |
| 12 | 0.1415 | 0.1897 |
| 16 | 0.1476 | 0.1942 |
| 20 | 0.1546 | 0.2025 |
| 24 | 0.1638 | 0.2124 |
| 28 | 0.1735 | 0.2241 |
| 32 | 0.1847 | 0.2369 |
| 36 | 0.1962 | 0.2509 |
| 40 | 0.2078 | 0.2657 |
| 44 | 0.2182 | 0.2793 |
| 48 | 0.2267 | 0.2921 |
| 52 | 0.2333 | 0.3031 |
| 56 | 0.2387 | 0.3122 |
| 60 | 0.2421 | 0.3201 |
| 64 | 0.2444 | 0.3262 |
| 68 | 0.246 | 0.3315 |
| 72 | 0.2475 | 0.3356 |
| 76 | 0.2483 | 0.339 |
| 80 | 0.2491 | 0.3421 |
| 84 | 0.2498 | 0.3443 |
| 88 | 0.2502 | 0.3462 |
| 92 | 0.2506 | 0.347 |
| 96 | 0.251 | 0.3477 |
| 100 | 0.2514 | 0.3481 |
| 104 | 0.2514 | 0.3485 |
| 108 | 0.2518 | 0.3489 |
| 112 | 0.2518 | 0.3489 |
| 116 | 0.2518 | 0.3489 |
| 120 | 0.2522 | 0.3492 |
| 124 | 0.2522 | 0.3492 |
| 128 | 0.2525 | 0.3489 |
| 132 | 0.2529 | 0.3489 |
| 136 | 0.2529 | 0.3489 |
| 140 | 0.2529 | 0.3489 |
| 144 | 0.2529 | 0.3489 |
| 148 | 0.2529 | 0.3489 |
| 152 | 0.2533 | 0.3492 |
| 156 | 0.2533 | 0.3489 |
| 160 | 0.2533 | 0.3489 |
| 164 | 0.2533 | 0.3485 |
| 168 | 0.2537 | 0.3485 |
| 172 | 0.2537 | 0.3485 |
| 176 | 0.2537 | 0.3485 |
| 180 | 0.2537 | 0.3485 |
| 184 | 0.2541 | 0.3485 |
| 188 | 0.2545 | 0.3485 |
| 192 | 0.2541 | 0.3485 |
| 196 | 0.2541 | 0.3485 |
| 200 | 0.2541 | 0.3481 |
| 204 | 0.2533 | 0.3485 |
| 208 | 0.2541 | 0.3481 |

Bioassays Employing Activated Partial Thromboplastin

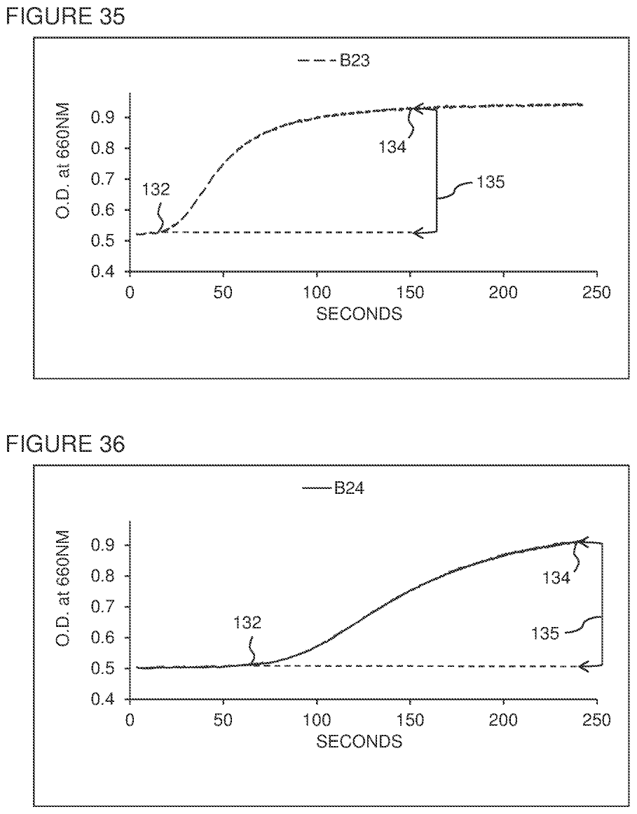

The results of different bioassays B23-B27 according to methods of the present invention employing varying matrixes for various microparticle dilutions are graphically illustrated in FIGS. 35® 38 and a discussion of each is provided below. Each of these bioassays B23-B27 employed an activated partial thromboplastin reagent. Specifically, the selected reagents 80 for these bioassays were activated partial thromboplastin, and CaCl₂) at the amounts listed in each of the protocols below.

B23-B24 Bioassays for Intrinsic Pathway

Turning now to FIGS. 35 and 36 which graphically illustrate the results of Activated Partial Thromboplastin Time (APPT) bioassays B23, B24 which measure the intrinsic pathway of coagulation, single step. Specifically, FIG. 35 illustrates the results of measurements of optical density versus time in seconds for a first APTT bioassay B23 of the present inventive method of a normal control. FIG. 36 then illustrates the results of measurements of optical density versus time in seconds for a a second APTT bioassay B24 of an abnormal control according to the present inventive method.

Both bioassays B23, B24 employed microparticle dilutions 11 having 0.392% weight per volume microparticles 24 with sulfate surface groups having a diameter of 110 nm. A drying matrix as discussed above was employed having 20 uL of 0.005 M CaCl₂). In another well, 100 uL of APTT-XL reagent was diluted with 150 uL of distilled water. The general methods for each of these bioassays B23-B24 were identical and generally prepared as discussed above with reference to FIG. 10.

As shown in FIG. 35, for the B23 assay, the clotting can be seen to begin 132 at about 13 seconds having an OD value of 0.5524, and clotting can be seen to be finished 134 at about 150 seconds having an OD value of 0.9315, with a change in absorption 135 over time being a change in optical density value having an OD value difference of 0.3791.

As shown in FIG. 36, for the B24 assay, the clotting can be seen to begin 132 at about 65 seconds having an OD value of 0.5111, and clotting can be seen to be finished 134 at about 240 seconds having an OD value of 0.9097, with a change in absorption 135 over time being a change in optical density value having an OD value difference of 0.3986.

The protocol for the bioassays B23, B24 is provided in Table 19 below:

TABLE 18
Protocol for Bioassays 23, 24

| Volume | Component |
|---|---|
| 30 uL | Diluted particles |
| 20 uL | Selected Matrix |
| 5 uL | Sample |
| 250 uL | APTT-XL Reagent w/ DH20 |

Table 19 shows the data of the optical density using a kinetic mode at 660 nm over three minutes for the first bioassay B23 with normal control and for the second bioassay B24 with abnormal control:

TABLE 19
Optical Density over Time

| SECONDS | B23 | B24 |
|---|---|---|
| 3.7 | 0.5219 | 0.5041 |
| 7.7 | 0.5229 | 0.5004 |
| 11.7 | 0.5268 | 0.5033 |
| 15.7 | 0.5263 | 0.5053 |
| 19.7 | 0.5368 | 0.5024 |
| 23.7 | 0.5522 | 0.504 |
| 27.7 | 0.5702 | 0.5031 |
| 31.7 | 0.597 | 0.5036 |
| 35.7 | 0.6318 | 0.5031 |
| 39.7 | 0.6656 | 0.5034 |
| 43.7 | 0.7008 | 0.505 |
| 47.7 | 0.7333 | 0.5037 |
| 51.7 | 0.7598 | 0.5059 |
| 55.7 | 0.7862 | 0.5074 |
| 59.7 | 0.8061 | 0.5101 |
| 63.7 | 0.8187 | 0.5109 |
| 67.7 | 0.8336 | 0.5135 |
| 71.7 | 0.8508 | 0.5162 |
| 75.7 | 0.8596 | 0.5185 |
| 79.7 | 0.8682 | 0.5256 |
| 83.7 | 0.8774 | 0.5309 |
| 87.7 | 0.8833 | 0.5396 |
| 91.7 | 0.8886 | 0.5482 |
| 95.7 | 0.893 | 0.5575 |
| 99.7 | 0.8998 | 0.5699 |
| 103.7 | 0.9037 | 0.5827 |
| 107.7 | 0.9072 | 0.5947 |
| 111.7 | 0.9099 | 0.6116 |
| 115.7 | 0.9126 | 0.6264 |
| 119.7 | 0.9155 | 0.6423 |
| 123.7 | 0.9175 | 0.6569 |
| 127.7 | 0.9206 | 0.6746 |
| 131.7 | 0.9213 | 0.6862 |
| 135.7 | 0.921 | 0.7014 |
| 139.7 | 0.9259 | 0.7156 |
| 143.7 | 0.9248 | 0.7294 |
| 147.7 | 0.9252 | 0.7442 |
| 151.7 | 0.9278 | 0.7563 |
| 155.7 | 0.929 | 0.7695 |
| 159.7 | 0.9322 | 0.779 |
| 163.7 | 0.9313 | 0.7907 |
| 167.7 | 0.9318 | 0.8 |
| 171.7 | 0.9309 | 0.8114 |
| 175.7 | 0.9352 | 0.822 |
| 179.7 | 0.9353 | 0.8286 |
| 183.7 | 0.9349 | 0.8365 |
| 187.7 | 0.9347 | 0.8426 |
| 191.7 | 0.9371 | 0.8518 |
| 195.7 | 0.9369 | 0.8589 |
| 199.7 | 0.9382 | 0.8667 |
| 203.7 | 0.9369 | 0.8726 |
| 207.7 | 0.9369 | 0.8764 |
| 211.7 | 0.9371 | 0.885 |
| 215.7 | 0.9457 | 0.8864 |
| 219.7 | 0.941 | 0.8898 |
| 223.7 | 0.9415 | 0.8932 |
| 227.7 | 0.9425 | 0.898 |
| 231.7 | 0.9404 | 0.9054 |
| 235.7 | 0.9401 | 0.9074 |
| 239.7 | 0.9454 | 0.9097 |
| 241.7 | 0.942 | 0.9159 |

B25-B27 Bioassays for 1-Stage APTT Based Factor

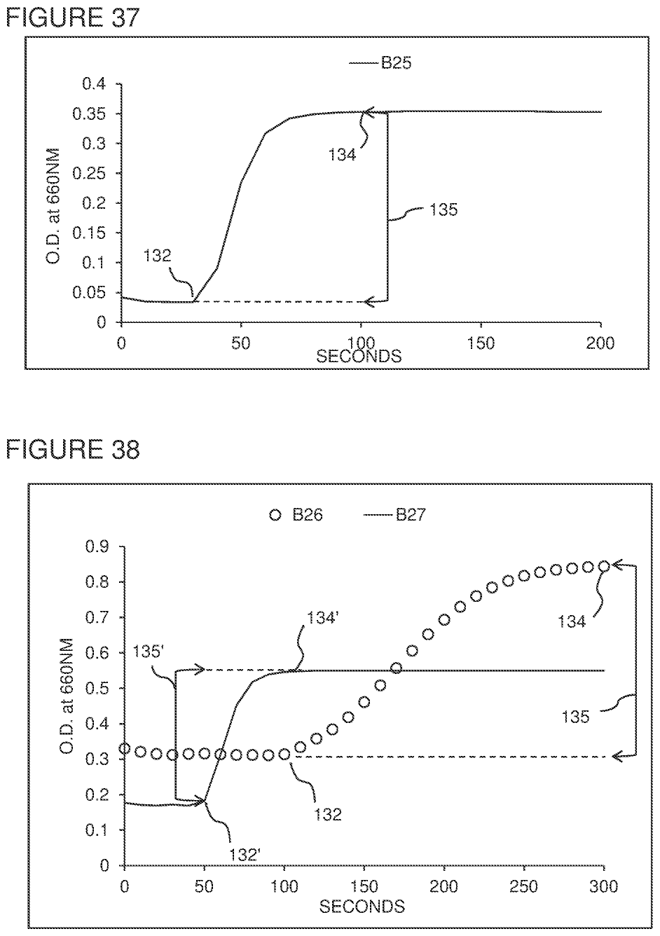

Specifically, FIGS. 37 and 38 graphically illustrate the results of three bioassays B25, B26, and B27 for 1-Stage APTT based Factors to measure the activity and activity level of the intrinsic pathway of coagulation. The first bioassay B25 uses APTT with normal plasma, the second bioassay B26 uses APTT with abnormal 99.9% Factor VIII deficient plasma, and the third bioassay B27 is an APTT 1-Stage Factor assay with a normal/abnormal mix of plasma.

Specifically, FIGS. 37 and 38 graphically illustrate the results of a bioassays B25, B26, B27 which employed a microparticle dilution 11 having 0.044% particle concentration of microparticles 24 with sulfate surface groups having a diameter of 110 nm. This dilution 11 had microparticles which were diluted with water by a ratio of 1 to 180. The optical density of the dilution 11 was measured at 660 nm to have an optical density value of 0.21. These bioassays each further employed a matrix 40 having 0.17M glycine at pH 10.0, and 1.0M NaCl.

Factors XII, XI, IX, VIII are measured directly with these assays. Additionally, Factors X, V, II are measured since they are involved in the common pathway cascade which lead to final clotting. Partial thromboplastin is formed from the sample by addition of common surface activators on Factor XII with the addition of calcium and phospholipid. Activators are, but not limited to, kaolin, celite, ellagic acid. One stage factor assays according to the present inventive method titer the samples individual factor level by comparing this level to a standard curve derived from dilutions of known factor deficient sample and a normal sample.

The general methods for bioassays B25, B26 were generally prepared as discussed above with reference to FIG. 10. The 1-Stage Factor bioassay B27 uses the same general method as discussed in FIG. 10, but additionally has a known amount of a single known factor deficient plasma dried in an additional well 6′″ to attenuate the response of the sample 60. Using a standard curve provided in the software, the level of actual response in the sample 60 is measured 116, corrected 118, and reported 119 according to the methods disclosed herein.

As shown in FIG. 37, for the B25 assay, the clotting can be seen to begin 132 at about 30 seconds having an OD value of 0.0340, and clotting can be seen to be finished 134 at about 100 seconds having an OD value of 0.3530, with a change in absorption 135 over time being a change in optical density value having an OD value difference of 0.3190.

As shown in FIG. 38, for the B26 assay, the clotting can be seen to begin 132 at about 100 seconds having an OD value of 0.3140, and clotting can be seen to be finished 134 at about 200 seconds having an OD value of 0.8440, with a change in absorption 135 over time being a change in optical density value having an OD value difference of 0.3986.

Also shown in FIG. 38, for the B27 assay, the clotting can be seen to begin 132′ at about 50 seconds having an OD value of 0.1820, and clotting can be seen to be finished 134′ at about 110 seconds having an OD value of 0.5480, with a change in absorption 135′ over time being a change in optical density value having an OD value difference of 0.3660.

The protocols for all three bioassays B25-B27 are provided in Table 20 below:

TABLE 20
Protocol for Bioassays B25-B27

| Volume uL's | Component |
|---|---|
| 490 uL | APTT activators in buffer, Ellagic acid |
| 10 uL | Sample |
| 25 uL | Microparticle Matrix |
| 25 uL | 0.02 M CaCl2 |
| +10 uL | known factor deficient plasma (B19 Only) |

Table 21 below shows the data of the corrected optical density results using a kinetic mode at 660 nm over five minutes for each of the above described bioassays B25-B27 according to the present invention:

TABLE 21
Optical Density over Time

| TIME, Secs | B25 | B26 | B27 |
|---|---|---|---|
| 0 | 0.042 | 0.33 | 0.177 |
| 10 | 0.035 | 0.321 | 0.171 |
| 20 | 0.034 | 0.315 | 0.169 |
| 30 | 0.034 | 0.312 | 0.172 |
| 40 | 0.091 | 0.315 | 0.169 |
| 50 | 0.235 | 0.316 | 0.182 |
| 60 | 0.317 | 0.314 | 0.312 |
| 70 | 0.342 | 0.312 | 0.451 |
| 80 | 0.349 | 0.312 | 0.518 |
| 90 | 0.352 | 0.311 | 0.539 |
| 100 | 0.353 | 0.314 | 0.546 |
| 110 | 0.353 | 0.334 | 0.548 |
| 120 | 0.354 | 0.358 | 0.55 |
| 130 | 0.354 | 0.384 | 0.55 |
| 140 | 0.354 | 0.418 | 0.55 |
| 150 | 0.354 | 0.461 | 0.55 |
| 160 | 0.354 | 0.508 | 0.55 |
| 170 | 0.354 | 0.557 | 0.55 |
| 180 | 0.353 | 0.606 | 0.55 |
| 190 | 0.353 | 0.652 | 0.55 |
| 200 | 0.353 | 0.693 | 0.55 |
| 210 | 0.353 | 0.73 | 0.55 |
| 220 | 0.353 | 0.76 | 0.55 |
| 230 | 0.353 | 0.784 | 0.55 |
| 240 | 0.353 | 0.803 | 0.55 |
| 250 | 0.353 | 0.817 | 0.55 |
| 260 | 0.353 | 0.827 | 0.55 |
| 270 | 0.353 | 0.834 | 0.55 |
| 280 | 0.354 | 0.838 | 0.55 |
| 290 | 0.354 | 0.842 | 0.55 |
| 300 | 0.354 | 0.844 | 0.55 |

### LIST OF REFERENCED ELEMENTS

The following reference numbers are adhered to within the specification to refer to those referenced elements within the drawings of the present application.

| | | | |
|---|---|---|---|
| 1 | Analyzer | 61 | Citrated blood |
| 2 | Cartridge | 62 | Whole blood |
| 3 | Barcode | 63 | Plasma |
| 4 | Fingerstick | 64 | Mix |
| 5 | sterilized pipette | 65 | Amount |
| 6 | Wells | 68 | Microparticle Mixture |
| 7 | Cuvette | 70 | Temperature |
| 8 | Sampler | 80 | Reagent/Activation |
| 9 | Main Packaging | | Agent |
| 10 | Microparticle Matrix | 82 | Thromboplastin |
| 11 | Microparticle Dilution | 84 | APTT |
| 18 | Functional group type | 86 | Thrombin |
| 20 | Plain | 88 | Ellagic Acid |
| 22 | Amidine | 90 | Diluent |
| 24 | Sulfate | 92 | CaCL2 |
| 26 | Size | 100 | Method |
| 28 | % weight by volume | 102 | Selecting |
| 30 | dilution ratio | | Cartridge/Bioassay |
| 31 | Optical Density value | 103 | Scanning cartridge with |
| 32 | Amount | | analyzer |
| 33 | Diluent | 104 | Obtaining blood sample |
| 40 | Matrix | 105 | Performing fingerstick |
| 42 | Wet matrix | 106 | Removing capillary |
| 44 | Drying Matrix | | sampler provided with |
| 46 | Carbohydrates | | cartridge |
| 47 | Glycine | 107 | Touching sampler to |
| 48 | Amount | | blood, filling sampler |
| 50 | acidity | | with sample |
| 52 | NaCl | 108 | Replacing sampler in |
| 54 | PEG | | cartridge |
| 56 | TWEEN | 109 | Inserting cartridge |
| 60 | Blood Sample | | within analyzer |
| 110 | Automated Bioassay | 126 | Corrected Clot Time |
| | Process Steps | 130 | Optical density |
| 112 | Adding component | 132 | Clotting begins |
| 114 | Mixing/Stirring | 134 | Clotting ends |
| | Components | 135 | Change in absorption = |
| 115 | Incubating | | change in optical |
| 116 | Measuring | | density over time |
| 118 | Correcting | 136 | Uncorrected PT |
| 119 | Reporting Results | 137 | HGB relationship |
| 120 | Adjusting | 138 | Corrected PT |
| 121 | Mixture | 140 | OD value detected at |
| 122 | Solution | | 530 nm |
| 123 | Commixture | 141 | Predetermined |
| 124 | Optically Detected clot | | relationship of OD |
| | formation | | value to HGB |
| 125 | Optically Detected | 142 | Specific HGB Level |
| | Hemoglobin Levels | | |

### CONCLUSION

Although the preferred embodiments of the present invention have been described herein, the above description is merely illustrative. Further modification of the invention herein disclosed will occur to those skilled in the respective arts and all such modifications are deemed to be within the scope of the invention as defined by the appended claims.

1. A disposable bioassay diagnostic cartridge for monitoring anticoagulant activity, the disposable cartridge comprising:
a first well holding an amount of matrix, the matrix being either a drying matrix or a liquid matrix;
a second well holding a plurality of microparticles, wherein the plurality of microparticles are uncoated latex with at least one surface type, and the at least one surface type being chosen from a group consisting of unreacted plain, sulfate, carboxylate, and amidine; and
a third well comprising an amount of an activation agent, the activation agent being chosen from a group consisting of thromboplastin, thrombin, ellagic acid, activated partial thromboplastin, Factor II, Factor VII, Factor I, Factor X, Factor XII, activated protein C, snake venom, negatively charged phospholipids, calcium ions, tissue factor, silica, koalin, and celite.

2. The disposable cartridge according to claim 1, wherein the matrix further comprises at least one of NaCl, PEG, TWEEN, carbohydrate, and CaCl2.

3. The disposable cartridge according to claim 1, the disposable cartridge further comprising an integrated cuvette capable of facilitating at least two optical detection readings.

4. The disposable cartridge according to claim 1, the disposable cartridge further comprising an integrated cuvette having a first wall capable of facilitating a first optical detection reading via a first LED at 530 nm; and a second wall capable of facilitating a second optical detection reading via a second LED at 660 nm.

5. The disposable cartridge according to claim 1, the disposable cartridge further comprising an integrated cuvette having first and second walls capable of facilitating a first optical detection reading via a first LED at 530 nm, and a second optical detection reading via a second LED at 660 nm.

6. (canceled)

7. A coagulation bioassay comprising:
a matrix, the matrix comprising at least one of glycine, sodium chloride, and 1% simethicone;
a plurality of microparticles suspended within the matrix, and wherein the plurality of microparticles are uncoated latex having at least one surface type; and
an amount of activation agent, the activation agent being chosen from a group consisting of thromboplastin, thrombin, ellagic acid, activated partial thromboplastin, Factor II, Factor VII, Factor I, Factor X, Factor XII, activated protein C, snake venom, negatively charged phospholipids, calcium ions, tissue factor, silica, koalin, and celite.

8. The coagulation bioassay according to claim 7, wherein the at least one surface type is selected from a group consisting of plain, sulfate, amidine, and carboxylate.

9. The coagulation bioassay according to claim 7, wherein the matrix is one of a drying matrix and a liquid matrix.

10. The coagulation bioassay according to claim 7, wherein each of the plurality of microparticles have a diameter of from about 10 nm to 150 nm.

11. The coagulation bioassay according to claim 7, wherein each of the plurality microparticles have a diameter in a range of from 90 nm to 110 nm.

12. The coagulation bioassay according to claim 7, wherein the plurality of microparticles are in a percent weight per volume solution selected from a group consisting of 0.006% weight per volume solution; 0.01% weight per volume solution; and 0.08% weight per volume solution.

13. A method of obtaining clotting time measurements using one of dilute, lysed whole blood, or plasma, the method comprising the steps of:
selecting a microparticle matrix having a matrix and a plurality of microparticles within the matrix, wherein the plurality of microparticles are uncoated latex with at least one surface type, and the at least one surface type chosen from a group consisting of unreacted plain, sulfate; carboxylate, and amidine chemical structures retaining activity;
using the microparticle matrix as a reagent with the one of dilute, lysed whole blood, or plasma; and
obtaining clotting time measurements of the one of dilute, lysed whole blood, or plasma; and
determining a hemoglobin level of the one of dilute, lysed whole blood, or plasma; and
correcting the clotting time measurements by adjusting for the hemoglobin level of the one of dilute, lysed whole blood, or plasma.

14. The method according to claim 13 further comprising: adding a separate activation agent to the reaction mixture to further activate natural clotting substrates in the one of dilute, lysed whole blood, or plasma.

15. The method according to claim 14 wherein the activation agent is selected from a group consisting of thromboplastin, thrombin, ellagic acid, activated partial thromboplastin, Factor II, Factor VII, Factor I, Factor X, Factor XII, activated protein C, snake venom, negatively charged phospholipids, calcium ions, tissue factor, silica, koalin, and celite.

16. The method according to claim 13 wherein obtaining the clotting time measurements of the one of dilute; lysed whole blood, or plasma further comprises repeatedly measuring an optical density of the one of dilute, lysed whole blood, or plasma, at a first wavelength over a period of time.

17. The method according to claim 16 wherein determining the hemoglobin level of the one of dilute, lysed whole blood, or plasma, comprises measuring an optical density of the one of dilute, lysed whole blood, or plasma, at a second wavelength.

18. The method according to claim 17 wherein the first wavelength is in a range of between 620 nm to 700 nm, and the second wavelength is in a range of between 500 nm to 550 nm.

19. The method according to claim 13 wherein the step of obtaining clotting time measurements of the one of dilute, lysed whole blood, or plasma further comprising obtaining an optical density value difference of at least 0.08.

20. The method according to claim 13 wherein the matrix is a liquid matrix and further comprises 0.17M glycine at pH 10.0, 1.29M NaCl, and 1% simethicone diluted with injection grade purified water.
